(19) United States
(12) Patent Application Publication
(10) Pub. No.: US 2022/0081707 A1
(43) Pub. Date: Mar. 17, 2022

(54) DIAGNOSTIC ASSAY FOR A STRAIN OF NEISSERIA MENINGITIDIS

(71) Applicant: THE TRUSTEES OF INDIANA UNIVERSITY, Bloomington, IN (US)

(72) Inventors: Aaron C. ERMEL, Indianapolis, IN (US); David E. NELSON, Zionsville, IN (US); Evelyn C. TOH, Zionsville, IN (US); James A. WILLIAMS, Noblesville, IN (US); Brahim QADADRI, Indianapolis, IN (US)

(21) Appl. No.: 17/262,012

(22) PCT Filed: Jul. 25, 2019
(86) PCT No.: PCT/US2019/043385

§ 371 Date: Jan. 21, 2021

Related U.S. Application Data
(60) Provisional application No. 62/703,080, filed on Jul. 25, 2018.

(51) Int. Cl. C12Q 1/689; C12Q 1/6827; C12Q 1/6806; C12Q 1/686

(52) U.S. Cl. CPC C12Q 1/689; C12Q 1/6827; C12R 2001/36; C12Q 1/686; C12Q 1/6806

(57) ABSTRACT

Methods for detecting and diagnosing a patient with urethritis are described. Methods for detection of a strain of urethrotropic Neisseria meningitides and compositions for performing the method are provided.

### CROSS REFERENCE TO RELATED APPLICATIONS

This application claims the benefit of U.S. Provisional Patent Application No. 62/703,080 filed on Jul. 25, 2018, the disclosure of which is hereby expressly incorporated by reference in its entirety.

### GOVERNMENT SUPPORT CLAUSE

This invention was made with government support under TR001108 awarded by the National Institutes of Health. The government has certain rights in the invention.

### TECHNICAL FIELD

The present disclosure relates to compositions and methods for detecting a strain of Neisseria meningitidis. The disclosure also relates to probes, primers, and methods of using those probes and primers to detect a strain of urethrotropic Neisseria meningitidis.

### BACKGROUND AND SUMMARY OF THE DISCLOSURE

Urethritis is clinically diagnosed by the presence of polymorphonuclear cells (PMNs) in male urethral smear or urine specimens and is often, but not always, associated with urethral and urinary symptoms. Millions of cases of urethritis occur in men in the United States each year, and urethritis-related symptoms are one of the most common reasons men under 50 seek clinical care. Infections with a number of sexually transmitted bacterial, viral, and protozoal pathogens can elicit urethritis, but many cases are idiopathic. Sexually transmitted bacterial pathogens including Chlamydia trachomatis (CT) and Neisseria gonorrhoeae (NG) are responsible for over half of all urethritis cases in US men. Previously, local public health clinics in Indianapolis, Ohio, Atlanta, and other major US cities began to notice an increased frequency of discordance between the nucleic acid amplification test (NAAT) results for NG and corresponding urethral Gram's stain smear results. By performing microbial culture, it was observed that many of these specimens grew Neisseria meningitidis (NM) instead of NG. Retrospective analysis of presumptive NG cases from a local clinic also confirmed this finding. Thus, a new clade of urethral Neisseria meningitidis (uNM) was identified. These urethrotropic NM (uMN) isolates were subsequently found to have acquired two genes from Neisseria gonorrhoeae (NG) that enables it to thrive in anaerobic environments. This urethrotropic clade of NM is indistinguishable from either commensal strains of NM or NG by conventional microscopy. There are currently no available molecular tests for uNM.

Urethritis is usually stratified in specialized sexually transmitted infection (STI) clinics based upon the results of a urethral Gram stain smear (GSS). The presence of Gram negative intracellular diplococci (GNID) in the GSS is highly sensitive and specific for N. gonorrhoeae (NG), and these men are diagnosed with gonococcal urethritis (GU). Men in whom GNID are not observed in the GSS are diagnosed with non-gonococcal urethritis (NGU). When a point of care diagnostic test, such as gram stain, is not available, diagnosis of NGU is often made on the basis of the results of a negative nucleic acid amplification test (NAAT) for NG. Chlamydia trachomatis (CT), Mycoplasma genitalium (MG), Trichomonas vaginalis (TV) are common causes of NGU, although other pathogens may cause this syndrome and many NGU cases are idiopathic. Sensitive and specific NAATs are available for some pathogens that cause NGU. However, syndromic management of urethritis based on GSS findings is the norm, and identification of NGU-associated pathogens, when attempted, is usually retrospective.

Neisseria meningitidis (NM) is best known for its ability to invade and cause meningitis and septicemia, although asymptomatic nasopharyngeal NM colonization is common. NM has occasionally been linked to single cases and clusters of cases of non-invasive urethritis. Until recently, none of these non-invasive NM-associated disease outbreaks was sustained.

STI clinics in the Midwest US have noted an increased frequency of GNID positive NG NAAT negative male urethritis cases. Urethral specimens from these men grew NM, and whole genome sequencing revealed that these isolates were members of a new, and nearly clonal, sub-group of strains in the ST-11 clonal complex, sub-lineage 11.2 (ST-11 uNM). The ST-11 uNM isolates are a new paradigm because they are non-groupable due to an insertion element in the capsule biosynthesis locus, and can grow anaerobically due to acquisition of a region of the norB-aniA locus from NG. The emergence of uNM ST-11 is alarming because this clade diverged from invasive ST-11 NM strains recently, and its pathogenic potential and epidemiology have not been completely defined. ST-11 uNM sub-lineages have acquired DNA from NG on multiple occasions since their recent divergence from an invasive ST-11 parent, so there is concern that the ST-11 uNM strains could also acquire genes that confer resistance to antibiotics and host immune defenses from NG.

NAATs have supplanted cultivation for diagnosis of NG, CT, TV, and MG in most clinical settings, as culture isolation of these microorganisms is costly, requires controlled transport conditions, and FDA-approved NAATs are now widely available and offer increased sensitivity. A NAAT that can detect and differentiate uNM ST-11 isolates from other NM strains and NG have not been reported. In prior studies, uNM ST-11 isolates were usually cultured from urogenital specimens in settings where there was a high degree of clinical suspicion due to discordant GSS and NG NAAT results, and then the identity of these isolates was confirmed by multi-locus sequence typing and or whole genome sequencing. Culture and whole genome sequencing are not available in many primary care settings where the majority of urethritis cases are seen, so uNM ST-11 prevalence has been interpreted from a handful of studies of male STI clinic attendees and retrospective analyses of banked isolates. Ultimately, not being able to identify this organism may adversely affect patient management and treatment decisions, especially in primary care settings where NAAT is likely the only test method used for diagnosis.

Herein described is a diagnostic method that both accurately identifies uNM, but can also distinguish between commensal NM strains and NG in clinical specimens. In one embodiment, a rapid real-time NAAT test is described for the uNM ST-11 clade strains using a SimpleProbe assay. The assay probe targets a single nucleotide polymorphism in norB that is conserved in uNM ST-11 clades strains, but absent in the other NM strains, NG, and commensal Neisseria species that can colonize the male urethra. Sensitivity and specificity of the assay were evaluated using urine and urogenital swab matrices spiked with urogenital pathogens and commensal urethral microorganisms. The assay also identified the only ST-11 uNM positive specimen in a collection of 241 male urine specimens that had been characterized by deep shotgun metagenomic sequencing. This uNM ST-11 clade SimpleProbe assay can be easily adapted for use with other types of clinical assays, is compatible with platforms used in contemporary STI diagnostic laboratories, and can be rapidly implemented for uNM ST-11 surveillance.

The various embodiments described in the numbered clauses below are applicable to any of the embodiments described in this “SUMMARY” section and the sections of the patent application titled “DETAILED DESCRIPTION OF ILLUSTRATIVE EMBODIMENTS” or “EXAMPLES” or in the “CLAIMS” appended to this application:

1. A method of detecting a urethrotropic strain of Neisseria meningitidis comprising:
extracting and recovering bacterial DNA from a patient sample;
amplifying the bacterial DNA using a forward and reverse primer to produce an amplicon;
hybridizing a probe to the products to specifically identify a urethrotropic strain of Neisseria meningitidis; and
identifying a urethrotropic strain of Neisseria meningitidis in the patient sample.

2. The method of clause 1, wherein the DNA is amplified using PCR.

3. The method of clause 2, wherein the PCR is an asymmetric PCR.

4. The method of clause 2, wherein the PCR is real-time PCR.

5. The method of clause 1, wherein the probe is fluorescently labeled.

6. The method of clause 2 wherein the probe is selected from the group consisting of 5′-CGTCATCAGCGATACGCG-3′, 5′-GCGATACGCGCGTGAAAGCCAT-3′, and 5′-CGATATGCGCGTGAAAGCCAT-3′.

7. The method of clause 2, wherein the forward primer is selected from the group consisting of 5′-GCTTGGCCGATGAATACC-3′ and 5′-GCTGATGACGAAAGACGA-3′.

8. The method of clause 2, wherein the reverse primer is selected from the group consisting of 5′-CAATGAAAAACAACACAT-3′ and 5′-CGTAAACGCCGTGATAGT-3′.

9. The method of clause 1, wherein the difference in melting temperatures (Tm) between the products of the PCR reaction and the fluorescence produced during a melting curve analysis is determined.

10. The method of clause 1, further comprising quantifying the amount of urethrotropic Neisseria meningitidis (uNM) in the sample.

11. The method of clause 1, wherein a gene from Neisseria gonorrhoeae (NG) is detected to identify said urethrotropic Neisseria meningitidis (uNM).

12. The method of clause 1, wherein the norB gene is detected. 13. The method of clause 1, wherein the probe targets a single nucleotide polymorphism in the norB gene.

14. The method of clause 9, wherein the difference in melting temperature of the products detects a single nucleotide polymorphism in the norB gene.

15. The method of clause 1, wherein the patient sample is a patient body fluid selected from the group consisting of urine, seminal fluid, vaginal fluid, other reproductive tract secretions, lymph fluid, whole blood, serum, and plasma.

16. The method of clause 1, wherein the patient has urethritis.

17. The method of clause 1, wherein the strain of Neisseria meningitidis is not a commensal strain.

18. An isolated nucleic acid molecule comprising a sequence selected from the group consisting of 5′-CGTCATCAGCGATACGCG-3′, 5′-GCTGATGACGAAAGACGA-3′, 5′-GCTTGGCCGATGAATACC-3′, 5′-CAATGAAAAACAACACAT-3′ and 5′-CGTAAACGCCGTGATAGT-3′.

19. A kit comprising a purified nucleic acid comprising a sequence selected from 5′-GCTGATGACGAAAGACGA-3′, 5′-GCTTGGCCGATGAATACC-3′, 5′-CAATGAAAAACAACACAT-3′ and 5′-CGTAAACGCCGTGATAGT-3′, and a fluorescently labeled probe.

20. The kit of clause 19, wherein the fluorescently labeled probe comprises a DNA sequence selected from the group consisting of 5′-CGTCATCAGCGATACGCG-3′, 5′-GCGATACGCGCGTGAAAGCCAT-3′, and 5′-CGATATGCGCGTGAAAGCCAT-3′.

### BRIEF DESCRIPTION OF THE DRAWINGS

FIG. 1. A graphic representation of the melting curves generated using the single probe developed for this assay. The difference in melting temperatures is demonstrated by the curves labeled NM1 and NM2 corresponding to two locally derived urethrotropic NM isolates and the NG positive control, and another locally derived NG.

FIG. 2. Melting curves corresponding to two locally derived urethrotropic NM isolates NM1, NM2, the NG positive control, and another locally derived NG isolate showing the difference in melting temperatures. The shift in melt curve peaks are clearly distinguishable, and separated by approximately 10° C.

FIG. 3. A graphic representation of the amplification phase of the reaction preceding the melting curve analysis of the same isolates mentioned in FIGS. 1 and 2.

FIG. 4 Amplification curves generated when the SimpleProbe designed for this assay was tested against various pathogens that could also occupy the urogenital tract. The assay correctly distinguished the organisms classified as urethrotropic NM strains (labeled as NM plasmid control, NM 1 and NM2) and organisms containing the wild type NG (labeled as NG plasmid control, GC, n-Lactimica or Neisseria lactamica, Pcon). The assay was also negative for any amplification for unrelated organisms (labeled as Fam 18, N-cineria, N-Perflava, N-Subflava, HSV ½, TC 748, TC 1030, UV Serovar 5, TC 593, HPV 16 plasmid) demonstrating the high specificity of this assay.

FIG. 5. A graph showing Standard Curve Quantification.

FIG. 6. A graphic representation of the melting curve of the same organisms shown in FIG. 4, demonstrating the differences in the melting curves between the various pathogens tested.

FIG. 7 Amplification curves generated when the SimpleProbe assay was tested against various pathogens that could also occupy the urogenital tract. The two peaks were again separated by 10° C. and were appropriately identified as urethrotropic NM vs. NG or N. lactamica.

FIG. 8 Amplification curves generated when the SimpleProbe assay was tested against various dilutions of NG and NM.

FIG. 9 Amplification curves generated when the SimpleProbe assay was tested against the first 20 men in the IUMP cohort.

FIG. 10 Amplification curve of one IUMP participant who tested positive for uNM in a separate group of 20 men.

FIGS. 11 A, B, and C. The identification of an urethrotropic NM isolate is provided: (A) Endpoint Fluorescence Scatter Plot showing Fluorescence (610-645) vs. Fluorescence (540-580); (B) Fluorescence History showing Fluorescence (540-580) vs. Cycle number; and (C) Fluorescence History showing Fluorescence (610-645) vs. Cycle number.

FIGS. 12 A, B, and C. The assay does not trigger a positive result in the presence of other common urogenital pathogens. The specificity of the assay is shown: (A) Endpoint Fluorescence Scatter Plot showing Fluorescence (610-645) vs. Fluorescence (540-580); (B) Fluorescence History showing Fluorescence (540-580) vs. Cycle number; and (C) Fluorescence History showing Fluorescence (610-645) vs. Cycle number.

### DETAILED DESCRIPTION OF THE INVENTION

As used herein, “a” or “an” may mean one or more. As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

The present disclosure is generally related to methods for detecting a strain of Neisseria meningitidis (NM). Specifically, the methods are useful for the rapid detection of urethrotropic Neisseria meningitidis. Described herein is a SimpleProbe Real-Time PCR Assay for the rapid detection and identification of urethrotropic Neisseria meningitidis (uNM) clade isolates in clinical specimens.

The molecular assay described herein can differentiate between the commensal strains of NM of the urethra from the urethrotropic NM strains causing urethritis. This novel urethritis-associated NM was recently responsible for a clonal epidemic of NG nucleic acid amplification test (NAAT) negative urethritis that was identified in the Midwest and Southeast. In addition, the assays described herein can discriminate between urethrotropic NM and Neisseria gonorrhoeae (NG), making the assays extremely valuable as a diagnostic tool.

The prevalence of the urethrotropic NM and commensal strains are generally unknown in a patient population. Thus, described herein is an NM specific quantitative real-time PCR (qPCR) assay using TaqMan® technology for detecting the norB gene. In one embodiment, a norB gene is detected. In one embodiment, a metA gene is detected. This assay allows for a sensitive screening tool to detect commensal strains and the urethrotropic strains. This urethrotropic NM specific assay as herein described can be applied downstream to differentiate the commensal from the disease causing urethrotropic NM strains.

The assay disclosed herein can accurately identify urethrotropic NM, thus providing clinicians with pertinent information, and a method for monitoring this emerging pathogen. This assay is suitable for testing using low or high volume testing platforms, and can be a key component in a multiplex PCR diagnostic, where it is possible to identify multiple sexually transmitted disease pathogens in a single reaction.

Methods are provided in accordance with the present invention for the detection of urethrotropic Neisseria meningitidis (uNM). In one embodiment, the method comprises extracting and recovering DNA from a patient sample, amplifying the DNA to produce an amplicon, hybridizing a probe to the amplicon to specifically identify uNM. In one embodiment, uNM is detected in the patient sample. In one embodiment, the uNM probe sequence is 5′-CGTCATCAGCGATACGCG-3′. In one embodiment, for amplifying the DNA, the forward NM primer sequence is 5′-GCTTGGCCGATGAATACC-3′ and the reverse NM primer sequence is 5′-CGTAAACGCCGTGATAGT-3′.

In accordance with the invention, “patient” may refer to a human or an animal. Accordingly, the methods and compositions disclosed herein can be used for both human clinical medicine and veterinary applications. Thus, as described herein, a “patient” can be a human or, in the case of veterinary applications, the patient can be a laboratory, an agricultural, a domestic, or a wild animal. In various aspects, the patient can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale. In one embodiment, the sample is obtained from a patient. In one embodiment, the patient sample is a patient body fluid selected from the group consisting of urine, seminal fluid, vaginal fluid (e.g., from a vaginal swab), other reproductive tract secretions, lymph fluid, whole blood, serum, and plasma. In another embodiment, the sample is a clinical sample. The samples can be prepared for testing as described herein.

In one embodiment, PCR is used as an amplifying step with one forward and one reverse primer that hybridize specifically to the DNA segment of interest. In one embodiment, the PCR is an asymmetric PCR. In one embodiment, the PCR is real-time PCR. The PCR may further comprise steps of quantitative real-time PCR. In one embodiment, the primers amplify a DNA sequence in a urethrotropic NM (uNM) strain that has been acquired from Neisseria gonorrhoeae (NG). In one embodiment, the forward primer may comprise 5′-GCTTGGCCGATGAATACC-3′ or 5′-GCTGATGACGAAAGACGA-3′, or the forward primer may comprise a nucleic acid having about 90%, about 95%, 96%, 97%, 98%, or 99% homology to said sequence or complements thereof. In one embodiment, the reverse primer may comprise 5′-CAATGAAAAACAACACAT-3′ or 5′-CGTAAACGCCGTGATAGT-3′, or the reverse primer may comprise a nucleic acid having about 90%, about 95%, 96%, 97%, 98%, or 99% homology to said sequence or complements thereof. In one embodiment, real-time PCR combines amplification and simultaneous probe hybridization to achieve sensitive and specific detection of the target DNA.

In the methods herein described, DNA may be detected and/or quantified using any DNA detection method known in the art. In one embodiment, the nucleic acid may be detected using conventional polymerase chain reaction (PCR) methods. In one embodiment, the nucleic acid may be detected using conventional polymerase chain reaction (PCR) or quantitative PCR (qPCR)). As described herein, PCR techniques may be used to amplify specific, target DNA fragments from low quantities of source DNA or RNA (for example, after a reverse transcription step to produce complementary DNA (cDNA), or detection of small fragment ctDNAs in a sample). When performing conventional PCR, the final concentration of template is proportional to the starting copy number and the number of amplification cycles. In one embodiment, a given number of reactions is performed on a single sample and the result is an analysis of fragment sizes or, for quantitative real-time PCR (qPCR), the analysis is an estimate of the concentration of the target sequences in the reaction-based on the number of cycles required to reach a quantification cycle (Cq).

In one embodiment, asymmetric PCR is used. Asymmetric PCR is a variation of PCR used to preferentially amplify one strand of DNA more than the other strand. In one embodiment, asymmetric PCR may be used in sequencing and applications where hybridizing a probe to only one of the two complementary strands is described. For asymmetric PCR, an excess amount of primer is used for a chosen strand. Due to the slow (arithmetic) amplification later in the reaction (after the limiting primer has been used up) extra cycles of PCR may be used in some instances. In one embodiment, a limiting primer with a higher melting temperature than the excess primer may be used to maintain reaction efficiency as the limiting primer concentration decreases mid-reaction. In one embodiment, asymmetric PCR may be used to form single stranded DNA for use in the assay.

In various embodiments of the methods and compositions described herein, the probes and primers can be labeled with fluorescent compounds, radioactive isotopes, antigens, biotin-avidin, colorimetric compounds, or other labeling agents known to those of skill in the art, to allow detection and quantification of amplified DNA, such as by PCR, Real-Time PCR, and the like. In illustrative embodiments, the labels may include Fluorescein, Oregon Green 488, Oregon Green 500, Oregon Green 514, Pacific Blue, Rhodamine Green, Rhodamine Red, Texas Red, and/or Alexa Fluor dyes and the like.

For qPCR methods, a fluorescent reporter dye is used as an indirect measure of the amount of nucleic acid present during each amplification cycle. The increase in fluorescent signal is directly proportional to the quantity of exponentially accumulating PCR product molecules (amplicons) produced during the repeating phases of the reaction. Reporter molecules may be categorized as; double-stranded DNA (dsDNA) binding dyes, dyes conjugated to primers, or additional dye-conjugated oligonucleotides, referred to as probes. The use of a dsDNA-binding dye, such as SYBR® Green I, represents the simplest form of detection chemistry. When free in solution or with only single-stranded DNA (ssDNA) present, SYBR Green I dye emits light at low signal intensity. As the PCR progresses and the quantity of dsDNA increases, more dye binds to the amplicons and hence, the signal intensity increases. Alternatively, a probe (or combination of two depending on the detection chemistry) can add a level of detection specificity beyond the dsDNA-binding dye, since it binds to a specific region of the template that is located between the primers. The most commonly used probe format is the Dual-Labeled Probe (DLP; also referred to as a Hydrolysis or TaqMan® Probe). The DLP is an oligonucleotide with a 5′ fluorescent label, e.g., 6-FAM™ and a 3′ quenching molecule, such as one of the dark quenchers e.g., BHQ®1 or OQ™ (see Quantitative PCR and Digital PCR Detection Methods). These probes are designed to hybridize to the template between the two primers and are used in conjunction with a DNA polymerase that has 5′ to 3′ exonuclease activity.

In various embodiments described herein, primers and/or probes that are used for amplification of the target DNA are oligonucleotides from about ten to about one hundred, more typically from about ten to about thirty or about twenty to about twenty-five base pairs long, but any suitable sequence length can be used. In illustrative embodiments, the primers and probes may be double-stranded or single-stranded, but the primers and probes are typically single-stranded. The primers and probes described herein are capable of specific hybridization, under appropriate hybridization conditions (e.g., appropriate buffer, ionic strength, temperature, formamide, or MgCl₂ concentrations), to a region of the target DNA. The primers and probes described herein may be designed based on having a melting temperature within a certain range, and substantial complementarity to the target DNA. Methods for the design of primers and probes are described in Sambrook et al., “Molecular Cloning: A Laboratory Manual”, 3rd Edition, Cold Spring Harbor Laboratory Press, (2001), incorporated herein by reference. Also within the scope of the invention are nucleic acids complementary to the probes and primers described herein, and those that hybridize to the nucleic acids described herein or those that hybridize to their complements under highly stringent conditions. In some illustrative aspects, hybridization occurs along the full-length of the nucleic acid.

In some embodiments, also included are nucleic acid molecules having about 60%, about 70%, about 75%, about 80%, about 85%, about 90%, about 95%, at least 96%, at least 97%, at least 98%, or at least 99% homology to the probes and primers described herein. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software. In some embodiments, the percent identity can be determined along the full-length of the nucleic acid. As used herein, the term “complementary” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

Techniques for synthesizing the probes and primers described herein are well-known in the art and include chemical syntheses and recombinant methods. Such techniques are described in Sambrook et al., “Molecular Cloning: A Laboratory Manual”, 3rd Edition, Cold Spring Harbor Laboratory Press, (2001), incorporated herein by reference. Primers and probes can also be made commercially (e.g., CytoMol, Sunnyvale, Calif. or Integrated DNA Technologies, Skokie, Ill.). Techniques for purifying or isolating the probes and primers described herein are well-known in the art. Such techniques are described in Sambrook et al., “Molecular Cloning: A Laboratory Manual”, 3rd Edition, Cold Spring Harbor Laboratory Press, (2001), incorporated herein by reference. The primers and probes described herein can be analyzed by techniques known in the art, such as restriction enzyme analysis or sequencing, to determine if the sequence of the primers and probes is correct.

In one embodiment, the probe targets a sequence in a urethrotropic NM (uNM) strain that has been acquired from Neisseria gonorrhoeae (NG). In one embodiment, the probe is fluorescently labeled. This probe comprises a nucleic acid sequence having at least 90% identity to a portion of the norB gene. For example, the probe can comprise a nucleic acid or portion of a nucleic acid as described in NM 1 isolate GenBank ID NO: LXLA00000000; NM2 GenBank ID NO: LXLB00000000; or a nucleic acid sequence having at least 90% identity to a portion of the norB gene as described. In one embodiment, the probe may comprise 5′-CGTCATCAGCGATACGCG-3′, 5′-GCGATACGCGCGTGAAAGCCAT-3′, or 5′-CGATATGCGCGTGAAAGCCAT-3′, or the probe may comprise a nucleic acid having about 90%, about 95%, 96%, 97%, 98%, or 99% homology to said sequence or complements thereof. In another embodiment, the probe comprises a nucleic acid sequence having at least 90% identity to a portion of the metA gene. In one embodiment, the probe is fluorescently labeled.

In another embodiment, the difference in melting temperatures (Tm) between the products of the PCR reaction and the fluorescence produced during a melting curve analysis may be detected. In another embodiment, the method further comprises quantifying the amount of urethrotropic Neisseria meningitidis (uNM) in the sample. Further, the method provides for detecting the uNM. In one embodiment, detecting a difference in melting temperature can be achieved using a fluorescence signal. In another embodiment, quantifying the amount of uNM can be achieved using the fluorescence signal. In another embodiment, the method comprises detecting the norB gene. Further, the method includes detecting a single nucleotide polymorphism in the norB gene. In another embodiment, the method comprises detecting the metA gene. In another embodiment, the method includes detecting a mutation in the norB gene and/or the metA gene.

In another embodiment, the method further comprises heating the products produced from amplifying the DNA by PCR [e.g., an amplicon]. In this step, the products come in contact with the probe and are heated to about 56 degrees Celsius. Alternatively, the first heating occurs at about 50 degrees Celsius, about 51 degrees Celsius, about 52 degrees Celsius, about 53 degrees Celsius, about 54 degrees Celsius, about 55 degrees Celsius, about 56 degrees Celsius, about 57 degrees Celsius, about 58 degrees Celsius, about 59 degrees Celsius, or about 60 degrees Celsius. During and after this first heating step, the products interacting with the probe are analyzed. In a subsequent heating, the products and probe are heated to about 65 degrees Celsius and the probe anneals. Alternatively, the second heating occurs at about 60 degrees Celsius, about 61 degrees Celsius, about 62 degrees Celsius, about 63 degrees Celsius, to about 64 degrees Celsius, to about 65 degrees Celsius, about 66 degrees Celsius, about 67 degrees Celsius, about 68 degrees Celsius, about 69 degrees Celsius, or about 70 degrees Celsius. The method further includes evaluating the difference in signal during analysis between the heating of the products and probes to about 56 degrees Celsius and the heating of the products and probes to about 65 degrees Celsius. In another embodiment, a signal is quantified during and after the first and second heating of the products and probes. The signal detects the presence of uNM in the sample.

In one embodiment a method is provided to detect uNM by contacting a forward primer and reverse primer to a DNA sample, performing PCR on the sample, analyzing the product (e.g., DNA amplicon), contacting the product with a probe, heating the product, and detecting uNM in the sample.

In various embodiments, sample preparation (i.e., preparation to extract DNA from a patient or clinical sample) involves rupturing the cells (e.g., cells of the tissue or bacteria in patient body fluid) and isolating the bacterial DNA from the lysate. Techniques for rupturing cells and for isolation of DNA are well-known in the art. For example, cells may be ruptured by using a detergent or a solvent, such as phenol-chloroform. DNA may be separated from the lysate by physical methods including, but not limited to, centrifugation, pressure techniques, or by using a substance with affinity for DNA, such as, for example, silica beads. After sufficient washing, the isolated DNA may be suspended in either water or a buffer. In other embodiments, commercial kits are available, such as Qiagen™, Nuclisensm™, and Wizard™ (Promega), and Promegam™. Methods for isolating DNA are described in Sambrook et al., “Molecular Cloning: A Laboratory Manual”, 3rd Edition, Cold Spring Harbor Laboratory Press, (2001), incorporated herein by reference.

The disclosure further includes probes for detecting the norB gene or the metA gene. In one embodiment, the probe comprises about 15 to about 25 nucleotides, or about 18 to about 20 nucleotides. In one embodiment, the probe comprises a nucleic acid sequence having 90% identity to a portion of the norB gene. Further, the probe can comprise a reporting agent. In some embodiments, the reporting agent is a molecule that emits fluorescence. For example, the reporting agent may be Fluorescein, but other reporting agents could be, but not limited to, Cy3, Cy5, Texas Red, members of the Alexa Fluor series (350-750). Using a distinct reporting agent will allow for multiple probes to be used in the same reaction. In another embodiment, the probe further comprises a quenching element. A quenching agent may be but not limited to Iowa Black, Black Hole quencher, TAMRA. In one embodiment, the probe comprises Fluorescein-SPC-CGTCATCAGCGATACGCG-Phosphate. In one embodiment, the probe comprises TEX615-GCGATACGCGCGTGAAAGCCAT-3IAbQ or HEX-CGATATGCGCGTGAAAGCCAT-BHQ1. The complementary sequence to the probe is also considered part of the disclosure. Primers are generated for specifically detecting a segment of the norB gene in a DNA sample. In one embodiment, primers are generated for specifically detecting a segment of the norB gene or the metA gene in a DNA sample.

The primers and probes described herein are capable of specific hybridization, under appropriate hybridization conditions (e.g., appropriate buffer, ionic strength, temperature, formamide, and MgCl₂ concentrations), to a region of the target DNA. The primers and probes described herein are designed based on having a melting temperature within a certain range, and substantial complementarity to the target DNA.

The following examples are exemplary embodiments of the disclosure. One of ordinary skill in the art will understand that slight variations or substitutions may be made to achieve the same results. Those slight variations and substitutions are considered a part of the disclosure herein.

### EXAMPLES

### Example 1

### Assay

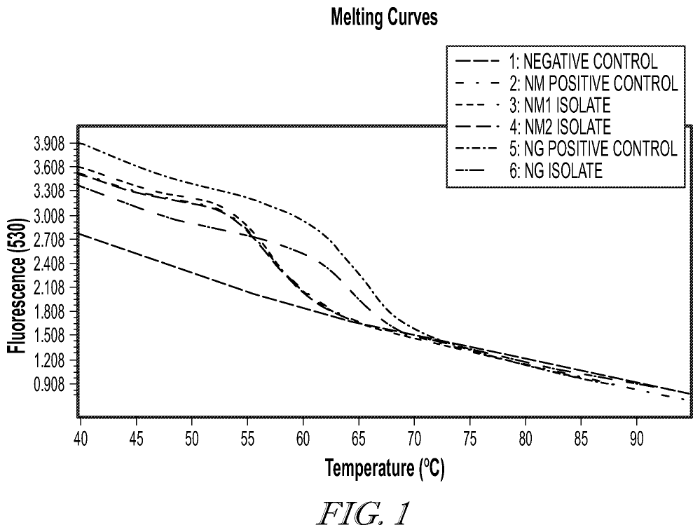

The assay uses an asymmetric polymerase chain reaction (PCR) coupled with a SimpleProbe designed to detect a single nucleotide polymorphism (SNP) within the transposed norB gene. The origin of this norB gene is thought to be from N. gonorrhoeae and is considered to be the wild-type allele. Specifically, in the urethrotropic (uNM) strain, there is a nucleotide change at bp 431 (G to A) in the wild-type norB sequence found in Neisseria gonorrhoeae. This SNP is highly conserved, and was identified in local NM isolates, and in isolates from other clinical sites across the US. Other candidate SNPs were considered, but were not uniformly present in every clinical isolate. The SimpleProbe method relies on a difference in melting temperatures (Tm) between the products of the PCR reaction and the fluorescence produced during the melting curve analysis. The Tm of the amplification product of this 117 bp region of the norB gene in NG and the uNM differ by approximately 10° C., as shown (see FIGS. 1, 2, and 3).

The qPCR assay uses a primer and probe labeled with a quencher and fluorescent probe set that is run using DNA polymerase with 5′ hydrolysis activity. During the reaction, a fluorescent signal is created if the probe binds to NM specific target DNA in the reaction. As amplification proceeds, the DNA polymerase hydrolyzes the probe and releases the fluorescent label, yielding a positive signal. Aside from identifying the presence of any uNM strain, this assay also quantifies the amount of bacteria present in the sample.

Simple probe
Fluorescein-SPC-CGTCATCAGCGATACGCG-Phosphate
Forward primer
5′-GCTTGGCCGATGAATACC-3′
Reverse primer
5′-CGTAAACGCCGTGATAGT-3′

Results: The assay was evaluated using both contrived samples and clinical samples (derived from the Bell Flower Clinic in Indianapolis, Ind.). The assay has successfully discriminated between urethrotropic and commensal NM strains, and between urethrotropic NM and NG. Therefore, this assay may be used as a diagnostic test to detect urethrotropic NM alone, or in combination, to detect NM and NG from the same clinical specimen in a single reaction.

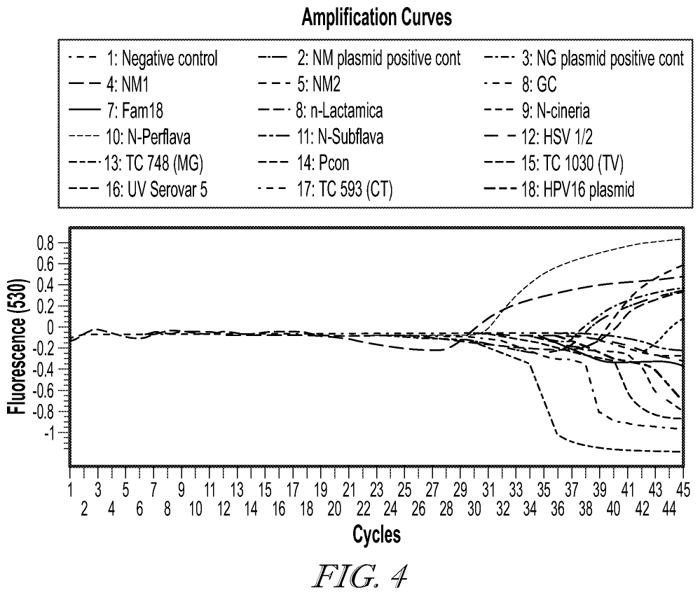

Experiments conducted in the CTSI designated Infectious Disease Core Laboratory have demonstrated high assay specificity and the ability to correctly identify urethrotropic NM from other commensal Neisseria species. FIGS. 4, 5, 6, and 7 show the results of experiments using several commensal Neisseria species and other pathogens that may be found in the urogenital tract. The limit of detection was also determined for both urine and vaginal swab matrix specimens. Other specimens used as input in the NM PCR reactions include residual processed specimens, previously extracted using an automated clinical analyzer. The assays described herein may be scaled up, e.g., to an automated system, that allows for high throughput testing.

The primer pair and probe were designed using the Roche Applied Science LightCycler Probe design software 2.0. The Fluorescein labeled probe was synthesized by FLUORESENTRIC (Park City, Utah, USA). The probe was labeled with Fluorescein and SPC at the 5′ end and phosphate at the 3′end.

An asymmetric PCR technique was used. The final concentration of the forward primer was 0.5 μM and 1.5 μM for the reverse primer. The final concentration of the probe was 0.2 μM. The LightCycler 480 Genotyping Master Mix (2×) was used. The MgCl₂ concentration was titrated for optimal reaction efficiency for the final concentration of MgCl₂ 3 mM. Two microliters of sample was used for a final total reaction volume of 20 μL.

The thermocycler conditions used for the reaction and melting curve analysis were as follows: initial denaturation at 95° C. for 10 min, followed by 45 cycles of 95° C. for 10 sec at a ramp rate of 20° C./s, 55° C. for 20 sec (single acquisition mode, ramp rate of 10° C./s, and 72° C. for 20 sec at a ramp rate of 20° C./s. The melting curve analysis was performed by raising the temperature to 95° C. for 0 sec (ramp rate of 20° C./s, followed by annealing step at 40° C. for 2 min (ramp rate of 1.5° C./s). As a final annealing step, the temperature was raised to 95° C. at a ramp rate of 0.1° C./s with continuous signal acquisition. The final cool down step was performed at 40° C. for 30 sec at a ramp rate 20° C./s (Table 4).

TABLE 4
Program

| Program Name | Program | | | | | |
|---|---|---|---|---|---|---|
| Cycles | 1 | Analysis Mode | None | | | |
| Target | Hold | Slope | Sec Target | Step size | Step Delay | Acquisition |
| (° C.) | (hr:mm:ss) | (° C./s) | (° C.) | (° C.) | (cycles) | Mode |
| 95 | 00:10:00 | 20 | 0 | 0 | 0 | None |
| Program Name | Program | | | | | |
| Cycles | 45 | Analysis Mode | Quantification | | | |
| Target | Hold | Slope | Sec Target | Step size | Step Delay | Acquisition |
| (° C.) | (hr:mm:ss) | (° C./s) | (° C.) | (° C.) | (cycles) | Mode |
| 96 | 00:00:10 | 20 | 0 | 0 | 0 | None |
| 55 | 00:00:20 | 10 | 0 | 0 | 0 | Single |
| 72 | 00:00:20 | 20 | 0 | 0 | 0 | None |
| Program Name | Program | | | | | |
| Cycles | 1 | Analysis Mode | Melting Curves | | | |
| Target | Hold | Slope | Sec Target | Step size | Step Delay | Acquisition |
| (° C.) | (hr:mm:ss) | (° C./s) | (° C.) | (° C.) | (cycles) | Mode |
| 95 | 00:00:00 | 20 | 0 | 0 | 0 | None |
| 40 | 00:02:00 | 1.5 | 0 | 0 | 0 | None |
| 95 | 00:00:00 | 0.1 | 0 | 0 | 0 | Continuous |
| Program Name | Program | | | | | |
| Cycles | 1 | Analysis Mode | None | | | |
| Target | Hold | Slope | Sec Target | Step size | Step Delay | Acquisition |
| (° C.) | (hr:mm:ss) | (° C./s) | (° C.) | (° C.) | (cycles) | Mode |
| 40 | 00:00:30 | 20 | 0 | 0 | 0 | None |

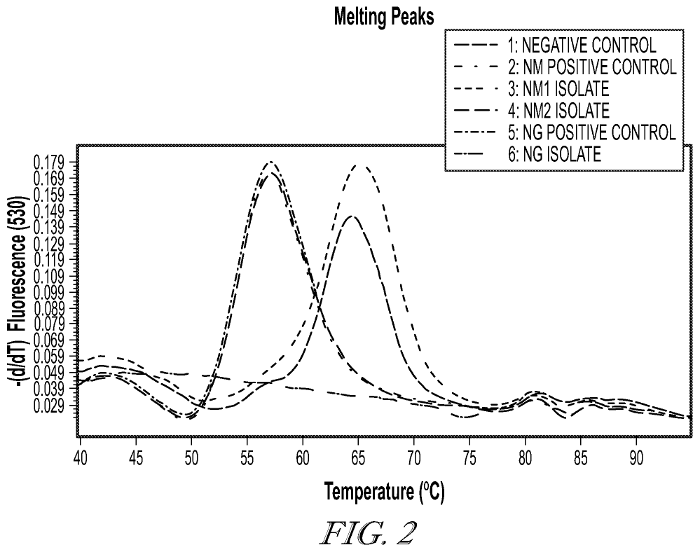

TABLE 5
Results for Melting Curve (FIG. 1) and Melting Peaks (FIG. 2) as shown.

| | | | Peak 1 | | | | Peak 2 | | | |
|---|---|---|---|---|---|---|---|---|---|---|
| Inc | Pos | Sample Name | Tm | Area | Wide | High | Tm | Area | Wide | High |
| [symbol] | 1 | NEGATIVE CONTROL | | | | | | | | |
| | 2 | NM POSITIVE CONTROL | 56.62 | 1.00 | 2.16 | 0.14 | | | | |
| | 3 | NM1 ISOLATE | 56.77 | 1.30 | [symbol] | 0.16 | | | | |
| | 4 | NM2 ISOLATE | 56.33 | 1.12 | 2.01 | 0.15 | | | | |
| | 5 | NG POSITIVE CONTROL | 66.20 | 1.23 | [symbol] | 0.15 | | | | |
| | 6 | NG ISOLATE | 64.39 | 0.83 | 2.25 | 0.12 | | | | |

[symbol]

indicates data missing or illegible when filed

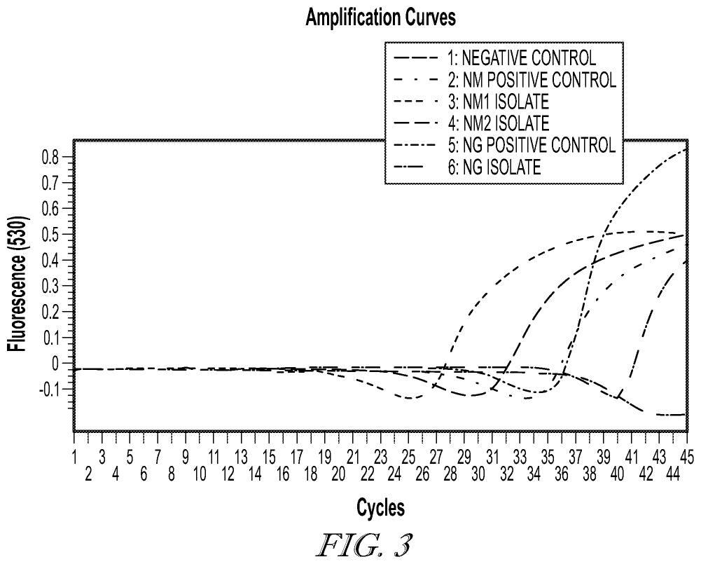

TABLE 6
Results for Amplification Curves (FIG. 3) as shown.

| Inc | Pos | Name | Type | CP | Concentratio | Standard |
|---|---|---|---|---|---|---|
| [symbol] | 1 | NEGATIVE CONTROL | UNKNOWN | | | |
| | 2 | NM POSITIVE CONTROL | UNKNOWN | [symbol] | | |
| | 3 | NM1 ISOLATE | UNKNOWN | [symbol] | | |
| | 4 | NM2 ISOLATE | UNKNOWN | [symbol] | | |
| | 5 | NG POSITIVE CONTROL | UNKNOWN | [symbol] | | |
| | 6 | NG ISOLATE | UNKNOWN | [symbol] | | |

[symbol]

indicates data missing or illegible when filed

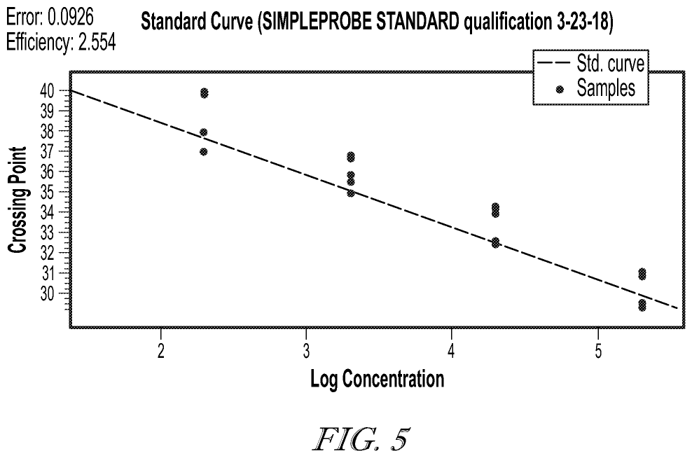

TABLE 7
Results for Amplifcation Curves (FIG. 4) and Standard Curve (FIG. 5) as
shown.

| Inc | Pos | Name | Type | CP | Concentratio | Standard |
|---|---|---|---|---|---|---|
| [symbol] | 1 | Negative control | Unknown | | | |
| | 2 | NM plasmid positive cont | Standard | 34.34 | [symbol] | 3.00E3 |
| | 3 | NG plasmid positive cont | Standard | 36.28 | [symbol] | 2.00E2 |
| | 4 | NM1 | Unknown | 26.23 | [1.00E7] | |
| | 5 | NM2 | Unknown | 34.03 | [symbol] | |
| | 6 | GC | Unknown | −40.00 | [−2.45E1] | |
| | 7 | Fam16 | Unknown | | | |
| | 8 | n- [symbol] | Unknown | 26.46 | [1.21E8] | |
| | 9 | N- [symbol] | Unknown | | | |
| | 10 | N- [symbol] | Unknown | | | |
| | 11 | N-Substance | Unknown | | | |
| | 12 | HPV 1/2 | Unknown | | | |
| | 13 | TC 746 (MG) | Unknown | | | |
| | 14 | [symbol] | Unknown | [symbol] | 4.11E2 | |
| | 15 | TC 1030 (TV) | Unknown | | | |
| | 16 | UV Sarovar 5 | Unknown | | | |
| | 17 | TC 593 (CT) | Unknown | [ [symbol] ] | [ [symbol] ] | |
| | 18 | HPV15 plasmid | Unknown | | | |

[symbol]

indicates data missing or illegible when filed

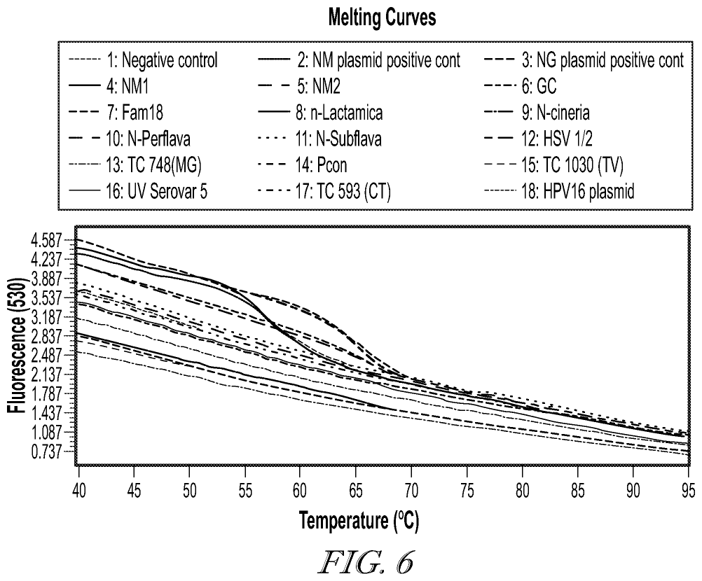

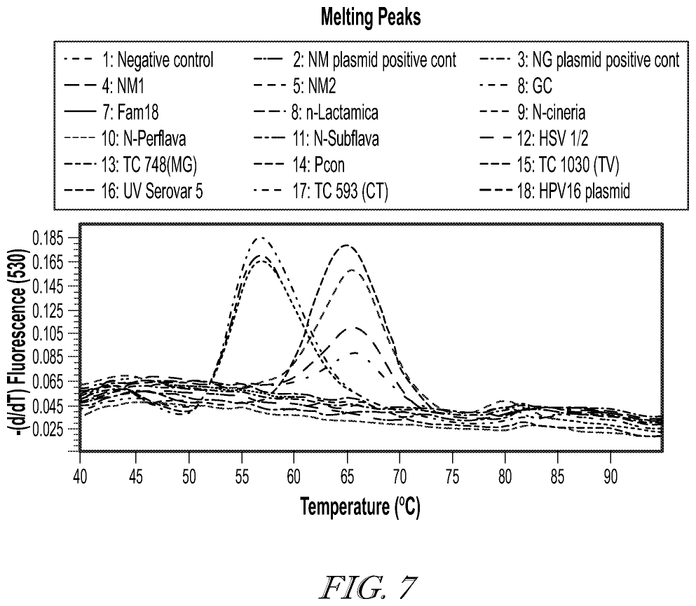

TABLE 8
Results for Melting Curves (FIG. 6) and Melting Peaks (FIG. 7) as shown.

| | | | Peak 1 | | | | Peak 2 | | | |
|---|---|---|---|---|---|---|---|---|---|---|
| Inc | Pos | Sample Name | Tm | Area | Wide | High | Tm | Area | Wide | High |
| | 1 | Negative control | | | | | | | | |
| | 2 | NM plasmid positive cont | 56.58 | 0.79 | 2.15 | 0.11 | | | | |
| | 3 | NG plasmid positive cont | 85.83 | 0.91 | 1.85 | 0.11 | | | | |
| | 4 | NM1 | [symbol] | 1.41 | 1.61 | 0.16 | | | | |
| | 5 | NM2 | [symbol] | 1.23 | 1.61 | 0.16 | | | | |
| | 6 | GC | 87.01 | 0.24 | 2.22 | 0.03 | | | | |
| | 7 | Fam16 | | | | | | | | |
| | 8 | n- [symbol] | [symbol] | 0.39 | 2.50 | 0.06 | 86.21 | 0.77 | 2.13 | 0.11 |
| | 9 | N- [symbol] | | | | | | | | |
| | 10 | N- [symbol] | | | | | | | | |
| | 11 | N-Substance | | | | | | | | |
| | 12 | HPV 1/2 | | | | | | | | |
| | 13 | TC 746 (MG) | | | | | | | | |
| | 14 | [symbol] | [symbol] | [symbol] | 2.10 | [symbol] | | | | |
| | 15 | TC 1030 (TV) | | | | | | | | |
| | 16 | UV Sarovar 5 | | | | | | | | |
| | 17 | TC 593 (CT) | | | | | | | | |
| | 18 | HPV15 plasmid | | | | | | | | |

[symbol]

indicates data missing or illegible when filed

### Example 2

### Bacterial Strains, Plasmids and DNA Templates

This study included a collection of anogenital pathogens and other Neisserial species obtained from American Type Culture Collection (ATCC) and other sources: N. meningitidis FAM18 (ATCC 700532D-5), N. cinerea (ATCC-14685) N. perflava (ATCC-14799), N. subflava (ATCC 49275), Chlamydia trachomatis (clinical isolate), Mycoplasma genitalium (clinical isolate), Trichomonas vaginalis (clinical isolate), and Ureaplasma urealyticum, (University of Alabama Birmingham). Plasmid and DNA templates included: HSV 1 and 2 control DNA (Acrometrix, Thermo Fisher Scientific Life.

### Example 3

### Collection and Characterization of Clinical Urines Used for Assay Validation

Males ≥18 years of age who presented to the Marion County Public Department of Health (MCPHD) Bellflower STI Clinic in Indianapolis, Ind. with acute urethral symptoms or as healthy controls without symptoms, and provided written consent to participate, were enrolled in idiopathic urethritis men's project (IUMP). A urethral swab from each participant was used to create a GSS to evaluate: (1) the presence of GNID and PMN/HPF. An aliquot of first-catch urine was also collected for uNM ST-11 testing. Men were diagnosed with NGU if they had ≥2 PMN/HPF by urethral GSS and/or urethral discharge on physical exam (N=127). Asymptomatic men without urethral discharge and <2 PMN's/HPF on GSS were identified as healthy controls (N=114). Total DNA from the urethral swabs was extracted and dual indexed sequencing libraries were constructed using the NexteraXT DNA Library preparation kit (Illumina, San Diego, Calif.). Sequencing libraries were pooled 12 per lane, and sequenced on the Illumina HiSeq 4000, generating 150 base paired-end sequences. Microorganism sequences were annotated using MetaPhlan2 (30). N. meningitidis sequences were detected in the urethral specimen from one man with idiopathic NGU, and presence of uNM ST-11 in the corresponding urine specimen of this man was confirmed by PCR amplification and sequencing of the norB-aniA locus.

### Example 4

### Simpleprobe uNM ST-11 Real-Time PCR Assay

A primer set was designed with Roche Applied Science Light Cycler Probe design software 2.0 to amplify a 117 bp segment of the 2,256 bp norB gene that contains the target SNP in position 431 in the uNM ST-11 reference isolate NM1 (NM1). A SimpleProbe probe was synthesized by and labeled with Fluorescein and SPC at the 5′ end and phosphate at the 3′ end, respectively (FLUORESENTRIC, Park City, Utah). Primer and probe sequences used in the asymmetric PCR are in Table 2. The PCR reaction mixtures contained LightCycler 480 Genotyping Master Mix (2×) (Roche Diagnostics, Indianapolis, Ind.), 0.5 μm of the forward primer (F-NG), 1.5 μM of the reverse primer (R-NG), 0.2 μM probe (P-NG), 5 jai DNA template, and 3 mM MgCl₂ in a total reaction volume of 29 μl. Amplification conditions were: 95° C. for 10 min (ramp rate 4.4° C./s), 45 cycles at 95° C. for 10 sec (ramp rate of 4.4° C./s), 55° C. for 20 sec in single acquisition mode (ramp rate 2.2° C./s), 72° C. for 20 sec (ramp rate 4.4° C./s). After amplification, melting curve analysis commenced by raising the temperature to 95° C. for 1 min (ramp rate 4.4° C./s), followed by annealing at 40° C. for 2 min (ramp rate 2.2° C./s). In the final annealing step, the temperature was raised to 90° C. (ramp rate 0.06° C./s) with continuous signal acquisition. Cool down was performed at 40° C. for 30 sec (ramp rate 2.2° C./s).

TABLE 1
Patient demographics showing number of specimens.

| Parameter | Male | |
|---|---|---|
| No. of urine samples | 241 | |
| Sample type | | Median Age (yr) |
| Urethritis participants | 127 | 30.1 |
| Healthy controls | 114 | 31.8 |

TABLE 2
Primer and probe sequences targeting
the uNM ST-11 and FAM18 norB.

| Primers and probes | Sequences |
|---|---|
| F-NG | 5′-GCTGATGACGAAAGACGA-3′* |
| F-NM1 | 5′-GCTTGGCCGATGAATACC-3′ |
| R-NG | 5′-CAATGAAAAACAACACAT-3′* |
| R-NM1 | 5′-CGTAAACGCCGTGATAGT-3′ |
| Probe-NG | Fluorescein-SPC- |
| | CGTCATCAGCGATACGCG-Phosphate |
| Probe-NM1 | Fluorescein-SPC- |
| | CGTCATCAGCGATACGCG-Phosphate |

*F-NG and R-NG primers were used to make the plasmid
control pNG.

TABLE 3
SimpleProbe assay results using control strains for identification of uNM.

| Strain | metA Taqman assay | uNM SimpleProbe assay |
|---|---|---|
| NG | − | − |
| NM-FAM18 | + | − |
| NM1 | + | + |
| NM2 | + | + |
| NM3 | + | + |
| NM4 | + | + |

### Example 5

### Determination of Analytical Sensitivity

The limit of detection (LOD) of the SimpleProbe uNM ST-11 real-time assay was determined using spiked mock urine and vaginal swab matrices that tested negative for sexually transmitted infections (STI). Serial dilutions of plasmids pNG-norB and pNM1-norB were added directly to mock urine and vaginal swab solutions, with the final plasmid concentrations ranging from 25 to 1000 copies/ml in the spiked urine matrices, and 250-4000 copies/ml in the spiked vaginal swab matrices. The contrived samples were processed, and DNA was extracted on the Roche Cobas 4800 automated extraction platform. To construct the control plasmids, 400 bp amplicons of norB encompassing the region containing the target SNP were PCR amplified from NM1 or NG genomic DNA. The amplicons were cloned into the pCR2.1-TOPO vector (Thermo Fisher Scientific Life Sciences, Waltham, Mass., USA) to create pNM1-norB and pNG-norB. The norB inserts in the final plasmids were confirmed by sequencing.

### Example 6

### Taqman Assay for Neisserial Meta

A TaqMan assay for the NM metA gene was adapted from a prior study (Truong D T, Franzosa E A, Tickle T L, Scholz M, Weingart G, Pasolli E, et al. MetaPhlAn2 for enhanced metagenomic taxonomic profiling. Nat Methods. 2015; 12(10):902-3). The assay was scaled up for use with the User Defined Workflow Software version 2.0 for the Roche 480Z platform.

### Example 7

### Assay Design and Validation Using Control Strains

At least two key genetic changes drove the emergence of the uNM ST-11 clade strain: disruption of the capsule locus by an insertion sequence (IS) element and acquisition of a region of the norB-aniA locus via a horizontal gene transfer event with NG. As herein described, the IS element in the capsule locus was not targeted because this region can recombine with other IS elements present in genomes of the uNM ST-11 isolates. 96% of the gonococcal norB gene remained intact in 204 uNM ST-11 clade strains surveyed retained. Alignment of the norB-ani locus of NM1, NG, NM FAM18, and other commensal Neisseria spp. revealed two nonsynonymous SNPs in norB, G431A and C1782T that were unique to NM1 (TABLE 9), and conserved in 95.2% of uNM ST-11 clade strains with a complete gonococcal norB gene.

The G431A SNP at position 431 is predicted to create a larger change in melting temperature than C1782T SNP, so a Simpleprobe assay was designed to target G431A using Applied Science Light Cycler Probe design software. A melting curve analysis was performed to assess if the Simpleprobe assay could differentiate the uNM ST-11 and NG norB alleles. NG norB melted between 65° C. and 67° C., whereas NM1 norB melted between 55° C. and 57° C. (FIG. 2).

The assay limit of detection (LOD) was assessed by spiking mock urine and vaginal swab matrices with serial dilutions of plasmids pNG-norB and pNM1-norB. Final plasmid concentrations ranged from 25 to 1000 copies/ml in the urine matrices, and 250-4000 copies/ml in the vaginal swab matrices. DNA was extracted from the contrived specimens using the Roche Cobas 4800 automated extraction platform and the SimpleProbe assay was performed on the LightCycler 2.0 and Roche Cobas 480Z light cycler platforms. Melting peaks were observed at the predicted temperatures, confirming that the SimpleProbe assay could discriminate NG and uNM ST-11 norB alleles in relevant matrices and on both PCR platforms. The limit of detection (LOD) was matrix and platform dependent. The lower LOD on the Light Cycler 2.0 platform was 50 copies/ml with urine and 500 copies/ml with vaginal swabs. On the Roche Cobas 480Z platform, the lower LOD was 100 copies/ml with urine and 1000 copies/ml with vaginal swabs.

A norB DNA sequence alignment by clustalW is shown below (Table 9). Two N. gonorrhoeae (NG) strains (FA1090, NCCP11945), one urethrotropic NM isolate (NM1), one Nm urethral isolate, and one N. lactamica (N1) strain were included in the alignment. Sequences unique exclusively to the urethrotropic NM are marked in bold and underlined. The target SNP for this assay is boxed.

TABLE 9
CLUSTAL W (1.83) multiple sequence alignment

| | |
|---|---|
| NG_NCCP11 945 | TTATTTGTCGCGGCCGAATACGATTTTAGTGGCTTGGATGGCAACGCAGATTGCACCGCCGATAAAGATTAAGTCGGGGG |
| NG_FA1090 | TTATTTGTCGCGGCCGAATACGATTTTAGTGGCTTGGATGGCAACGCAGATTGCACCGCCGATAAAGATTAAGTCGGGGG |
| uNM_NM1 | TTATTTGTCGCGGCCGAATACGATTTTAGTGGCTTGGATGGCAACGCAGATTGCACCGCCGATAAAGATTAAGTCGGGGG |
| NM_LNP26948 | TTATTTGTCGCGGCCGAATACGATTTTAGTGGCTTGGATGGCAACGCAGATTGCACCGCCGATAAAGACCAAGTCAGCTG |
| NM_FAM18 | TTATTTGTCGCGGCCGAATACGATTTTAGTGGCTTGGATGGCAACGCAGATTGCACCGCCGATAAAGACCAAGTCAGCTG |
| Nlac_020-06 | TTATTTGTCCCGGCCGAATACGATTTTAGTGGCTTGGATGGCAACGCAGATTGCACCGCCGATAAAGATCAAGTCGGCGG |
| | ********* ********************************************************** ***** * * |
| NG_NCCP11945 | CAGTGCGTACCCAGCGCAGGGTGTCGAGGATTTCCATTTGCAGGAATTCTTCGCTGCGGGCATACCACAAACCGTGCGTG |
| NG_FA1090 | CAGTGCGTACCCAGCGCAGGGTGTCGAGGATTTCCATTTGCAGGAATTCTTCGCTGCGGGCATACCACAAACCGTGCGTG |
| uNM_NM1 | CAGTGCGTACCCAGCGCAGGGTGTCGAGGATTTCCATTTGCAGGAATTCTTCGCTGCGGGCATACCACAAACCGTGCGTG |
| NM_LNP26948 | CCGTACGTACCCAACGCAAGGTGTCGAGGATTTCCATTTGCAGGAACTCTTCGCTGCGTGCATACCACAGGCGGTGCGTG |
| NM_FAM18 | CCGTACGTACCCAACGCAAGGTGTCGAGGATTTCCATTTGCAGGAACTCTTCGCTGCGTGCATACCACAGGCCGTGCGTG |
| Nlac_020-06 | CAGTACGAACCCAGCGCAGGGTGTCGAGGATTTCCATTTGCAGGAACTCTTCGCTGCGGGCATACCACAGACCGTGCGTG |
| | * ** ** ***** **** *************************** *********** ********** * ******* |
| NG_NCCP11945 | ATAGAGGCGTATGCCTGAATCACGCCGACAGGCAGCAGGCTGATGGCAATCATACCGACCAAGCCGCCGTTGAGCAGCCA |
| NG_FA1090 | ATAGAGGCGTATGCCTGAATCACGCCGACAGGCAGCAGGCTGATGGCAATCATACCGACCAAGCCGCCGTTGAGCAGCCA |
| uNM_NM1 | ATAGAGGCGTATGCCTGAATCACGCCGACAGGCAGCAGGCTGATGGCAATCATACCGACCAAGCCGCCGTTGAGCAGCCA |
| NM_LNP26948 | ATGGAGGCGTATGCCTGAATCACGCCAACCGGCAACAGGCTGATGGCAATCATACCGACCAAGCCGCCGTTGAGCAGCCA |
| NM_FAM18 | ATGGAGGCGTATGCCTGAATCACGCCAACCGGCAACAGGCTGATGGCAATCATACCGACCAAGCCGCCGTTGAGCAGCCA |
| Nlac_020-06 | ATGGAGGCGTATGCCTGAATCGCGCCGACAGGCAGCAGGCTGATGGCAATCATACCGACCAAGCCGCCGTTGAGCAACCA |
| | ** ****************** **** ** **** ***************************************** *** |
| NG_NCCP11945 | GAAGCCCCAAGTCATCAGTTTGTCGTCAAACCGCGCGTTCGGTTTCAAGTAGCGCGCAACCAACAATACGAAGCCCAATG |
| NG_FA1090 | GAAGCCCCAAGTCATCAGTTTGTCGTCAAACCGCGCGTTCGGTTTCAAGTAGCGCGCAACCAACAATACGAAGCCCAATG |
| uNM_NM1 | GAAGCCCCAAGTCATCAGTTTGTCGTCAAACCGCGCGTTCGGTTTCAAGTAGCGCGCAACCAACAATACGAAGCCCAATG |
| NM_LNP26948 | GAAGCCCCAAGTCATCAGTTTGTCGTCAAACTGCGCGTTCGGTTTCAAATAACGGGCAACCAGCAATACGAAGCCCAATG |
| NM_FAM18 | GAAGCCCCAAGTCATCAGTTTGTCGTCAAACTGCGCGTTCGGTTTCAAATAACGGGCAACCAGCAATACGAAGCCCAATG |
| Nlac_020-06 | GAAGCCCCAAGTCATCAGTTTGTCGTCAAACTGCGCGTTCGGTTTCAAGTAGCGTGCAACCAGCAATACGAAGCCCAATG |
| | ******************************* **************** ** ** ******* ***************** |
| NG_NCCP11945 | CCAAGAAACCGTACACACCGAACAAGGCGGCGTGCGCGTGAACGGCGGAAGTGTTCAAACCTTGGATATAGAACAGGGAA |
| NG_FA1090 | CCAAGAAACCGTACACACCGAACAAGGCGGCGTGCGCGTGAACGGCGGAAGTGTTCAAACCTTGGATATAGAACAGGGAA |
| uNM_NM1 | CCAAGAAACCGTACACACCGAACAAGGCGGCGTGCGCGTGAACGGCGGAAGTGTTCAAACCTTGGATATAGAACAGGGAA |
| NM_LNP26948 | CCAAGAAACCGTACACACCGAACAAGGCGGCGTGCGCATGAACAGCAGAAGTATTCAAACCTTGGATATAGAACAGGGAA |
| NM_FAM18 | CCAAGAAACCGTACACACCGAACAAGGCGGCGTGCGCATGAACAGCAGAAGTATTCAAACCTTGGATATAGAACAGGGAA |
| Nlac_020-06 | CCAAGAAACCGTACACACCGAACAAGGCGGCGTGCGCGTGAACGGCAGAAGTGTTCAAACCTTGGATATAGAACAGGGAA |
| | ************************************* ***** ** ***** *************************** |
| NG_NCCP11945 | ATCGGCGGATTGATCAGAAAGCCGAATACGCCGGCACCGATCATATTCCAAAAAGCGACTGCCACGAAGCACATCAGCGG |
| NG_FA1090 | ATCGGCGGATTGATCAGAAAGCCGAATACGCCGGCACCGATCATATTCCAAAAAGCGACTGCCACGAAGCACATCAGCGG |
| uNM_NM1 | ATCGGCGGATTGATCAGAAAGCCGAATACGCCGGCACCGATCATATTCCAAAAAGCGACTGCCACGAAGCACATTAGCGG |
| NM_LNP26948 | ATCGGCGGGTTAATCAGGAAACCGAATACACCGGCACCGATCATATTCCAAAAGGCGACTGCCACGAAGCACATCAGCGG |
| NM_FAM18 | ATCGGCGGGTTAATCAGGAAACCGAATACACCGGCACCGATCATATTCCAAAAGGCGACTGCCACGAAGCACATCAGCGG |
| Nlac_020-06 | ATCGGCGGATTGATCAGGAAGCCGAATACGCCGGCACCGATCATATTCCAAAAGGCGACTGCCACGAAGCACATCAGCGG |
| | ******** ** ***** ** ******** *********************** ******************** ***** |
| NG_NCCP11945 | CCAACGCAGGCGTTTCGCCCAGTCGGACAGGTGTTGGTAAGACCAGTGCTCGTATGCCTCGCGGCCCAGCAACACCAGCG |
| NG_FA1090 | CCAACGCAGGCGTTTCGCCCAGTCGGACAGGTGTTGGTAAGACCAGTGCTCGTATGCCTCGCGGCCCAGCAACACCAGCG |
| uNM_NM1 | CCAACGCAGGCGTTTCGCCCAGTCGGACAGGTGTTGGTAAGACCAGTGCTCGTATGCCTCGCGGCCCAGCAACACCAGCG |
| NM_LNP26948 | CCAACGCAGGCGTTTCGCCCAGTCGGACAGGTGTTGGTAAGACCAATGCTCGTATGCTTCACGGCCCAGCAACACCAGCG |
| NM_FAM18 | CCAACGCAGGCGTTTCGCCCAGTCGGACAGGTGTTGGTAAGACCAATGCTCGTATGCTTCACGGCCCAGCAACACCAGCG |
| Nlac_020-06 | CCAACGCAGGCGTTTCGCCCAGTCGGACAGATGTTGGTAAGACCAATGCTCGTATGCTTCACGACCCAGCAACACCAGCG |
| | ****************************** ************** *********** ** ** **************** |
| NG_NCCP11945 | GCACGACTTCCAAAGCGGAGAAGCAGGCGCCGATTGCCATAGAGGCGGAGGTAGAGCCGGAGAAGTACAGGTGGTGCAGC |
| NG_FA1090 | GCACGACTTCCAAAGCGGAGAAGCAGGCGCCGATTGCCATAGAGGCGGAGGTAGAGCCGGAGAAGTACAGGTGGTGCAGC |
| uNM_NM1 | GCACGACTTCCAAAGCGGAGAAGCAGGCGCCGATTGCCATAGAGGCGGAGGTAGAGCCGGAGAAGTACAGGTGGTGCAGC |
| NM_LNP26948 | GCACGACTTCCAAAGCGGAGAAGCAGGCACCGATTGCCATAGAGGCGGAGGTAGAGCCGGAGAAGTACAGGTGGTGCAGC |
| NM_FAM18 | GCACGACTTCCAAAGCGGAGAAGCAGGCACCGATTGCCATAGAGGCGGAGGTAGAGCCGGAGAAGTACAGGTGGTGCAGC |
| Nlac_020-06 | GCACGACTTCCAAAGCGGAGAAGCAGGCGCCGATTGCCATAGAGGCGGAGGTAGAGCCGGAGAAGTACAGGTGGTGCAGC |
| | **************************** *************************************************** |
| NG_NCCP11945 | GTGCCCGGAACGCCGCCCAACATAAAGATGGCGGCAGCGGCCAAAGTGGAGGCAGTGGCGGTACTGCGGCGGACAAAGCC |
| NG_FA1090 | GTGCCCGGAACGCCGCCCAACATAAAGATGGCGGCAGCGGCCAAAGTGGAGGCAGTGGCGGTACTGCGGCGGACAAAGCC |
| uNM_NM1 | GTGCCCGGAACGCCGCCCAACATAAAGATGGCGGCAGCGGCCAAAGTGGAGGCAGTGGCGGTACTGCGGCGGACAAAGCC |
| NM_LNP26948 | GTACCCGGAACGCCGCCCAACATAAAGATGGCGGCAGCGGCCAAAGTAGAAGCTGTGGCGGTACTGCGGCGGACAAAGCC |
| NM_FAM18 | GTACCCGGAACGCCGCCCAACATAAAGATGGCGGCAGCGGCCAGAGTAGAAGCTGTGGCGGTACTGCGGCGGACAAAGCC |
| Nlac_020-06 | GTACCGGGAACGCCGCCCAACATAAAGATGGCGGCAGCAGCCAAAGTGGAGGCAGTGGCGGTACTGCGGCGGACAAAGCC |
| | ** ** ******************************** **** *** ** ** ************************** |
| NG_NCCP11945 | CATATTGTAGAAGACAAAGGCAAAGGCGGCAGTGGCAAATACTTCAAAGAAGCCTTCCACCCACAGGTGGACCACCCACC |
| NG_FA1090 | CATATTGTAGAAGACAAAGGCAAAGGCGGCAGTGGCAAATACTTCAAAGAAGCCTTCCACCCACAGGTGGACCACCCACC |
| uNM_NM1 | CATATTGTAGAAGACAAAGGCAAAGGCGGCAGTGGCAAATACTTCAAAGAAGCCTTCCACCCACAGGTGGACCACCCACC |
| NM_LNP26948 | CATATTGTAGAAGACAAATGCGAAGGCGGCAGTGGCAAATACTTCAAAGAAGCCTTCCACCCACAGGTGAACCACCCACC |
| NM_FAM18 | CATATTGTAGAAGACAAAGGCAAAAGCGGCAGTGGCAAATACTTCAAAGAAGCCTTCCACCCACAGGTGAACCACCCACC |
| Nlac_020-06 | CATATTGTAGAAGACAAAGGCAAAGGCGGCAGTGGCAAATACTTCAAAGAAGCCTTCCACCCACAGGTGAACCACCCACC |
| | ****************** ** ** ******************************************** ********** |
| NG_NCCP11945 | AACGCCAGTATTCCATTACGGCAATCGGGGATTTTTCGCCATAGAACAGGCCCGGTGCGTAGAACACGCCCACGCCGACC |
| NG_FA1090 | AACGCCAGTATTCCATTACGGCAATCGGGGATTTTTCGCCATAGAACAGGCCCGGTGCGTAGAACACGCCCACGCCGACC |
| uNM_NM1 | AACGCCAGTATTCCATTACGGCAATCGGGGATTTTTCGCCATAGAACAGGCCCGGTGCGTAGAACACGCCCACGCCGACC |
| NM_LNP26948 | AACGCCAGTATTCCATTACGGCAATCGGGGATTTCTCGCCATAGAACAGTCCCGGTGCGTAGAATACGCCCACACCGACC |
| NM_FAM18 | AACGCCAGTATTCCATTACGGCAATCGGGGATTTCTCGCCATAGAACAGTCCCGGTGCGTAGAATACGCCCACACCGACC |
| Nlac_020-06 | AGCGCCAGTATTCCATTACGGCAATCGGGGATTTTTCGCCATAGAACAGTCCCGGTGCGTAGAACACGCCCACACCGACC |
| | * ******************************** ************** ************** ******** ****** |
| NG_NCCP11945 | ATAGAAGCGACAAAGATTGCCAGCAGGTTTTTGTCCACGCCTTTTTCTTTGAAGGCGGAAACCGTGCAGCGCAACATCAG |
| NG_FA1090 | ATAGAAGCGACAAAGATTGCCAGCAAGTTTTTGTCCACGCCTTTTTCTTTGAAGGCGGAAACCGTGCAGCGCAACATCAG |
| uNM_NM1 | ATAGAAGCGACAAAGATTGCCAGCAAGTTTTTGTCCACGCCTTTTTCTTTGAAGGCGGAAACCGTGCAGCGCAACATCAG |
| NM_LNP26948 | ATAGAAGCGACAAAGATTGCCAGCAGGTTTTTGTCCACGCCTTTTTCTTTGAAGGCGGAAACCGTGCAACGCAACATCAG |
| NM_FAM18 | ATAGAAGCGACAAAGATTGCCAGCAGGTTTTTGTCCACGCCTTTTTCTTTGAAGGCGGAAACCGTGCAACGCAACATCAG |
| Nlac_020-06 | ATAGAAGCGACAAAGATTGCCAGCAGGTTTTTATCCACGCCTTTTTCTTTGAAGGCGGAAACCGTGCAGCGCAACATCAG |
| | ************************* ****** *********************************** *********** |
| NG_NCCP11945 | GAACAGCCACAACAGCAGTCCGACCATCAAAAGGAGTTGCCAGAAACGTCCCAAATCGAGGTATTCGTAACCTTGGTGTC |
| NG_FA1090 | GAACAGCCACAACAGCAGTCCGACCATCAAAAGGAGTTGCCAGAAACGTCCCAAATCGAGGTATTCGTAACCTTGGTGTC |
| uNM_NM1 | GAACAGCCACAACAGCAGTCCGACCATCAAAAGGAGTTGCCAGAAACGTCCCAAATCGAGGTATTCGTAACCTTGGTGTC |
| NM_LNP26948 | GAACAGCCACAACAGCAGGCCGACCATCAGCAGGAGTTGCCAGAAACGTCCCAAATCGAGGTATTCGTAACCTTGGTGTC |
| NM_FAM18 | GAACAGCCACAACAGCAGGCCGACCATCAGCAGGAGTTGCCAGAAACGTCCCAAATCGAGGTATTCGTAACCTTGGTGTC |
| Nlac_020-06 | GAACAGCCACAACAGCAGGCCGACCATCAACAGGAGTTGCCAGAAACGTCCCAAATCGAGGTATTCGTAACCTTGGTGTC |
| | ****************** ********** ************************************************* |
| NG_NCCP11945 | CGAACCAGAAGTTAAATTCGGGGGGAAGGATGTGCGTCAACGCGAAGAAGTTGCCCGCGTAAGAACCGCCGACCACGATG |
| NG_FA1090 | CGAACCAGAAGTTAAATTCGGGAGGAAGGATGTGCGTCAACGCGAAGAAGTTGCCCGCGTAAGAACCGCCGACCACGATG |
| uNM_NM1 | CGAACCAGAAGTTAAATTCGGGAGGAAGGATGTGCGTCAACGCGAAGAAGTTGCCCGCGTAAGAACCGCCGACCACGATG |
| NM_LNP26948 | CGAACCAGAAGTTAAATTCCGGGGGAAGGATGTGCGTCAACGCGAAGAAGTTGCCCGCGTAAGAACCGCCGACCACGATG |
| NM_FAM18 | CGAACCAGAAGTTAAATTCCGGGGGAAGGATGTGCGTCAACGCGAAGAAGTTGCCCGCGTAAGAGCCGCCGACCACGATG |
| Nlac_020-06 | CGAACCAGAAGTTAAATTCGGGGGGAAGGATGTGCGTCAACGCGAAGAAGTTGCCCGCGTAAGAACCGCCGACCACGATA |
| | ******************* ** ***************************************** ************** |
| NG_NCCP11945 | AAGAGGGCGATATAGAGGAAGTTCACGCCTGCACGTTGGAACTTGGGATCTTTGCCGCCGTTGACAATCGGCGCGAGGAA |
| NG_FA1090 | AAGAGGGCGATATAGAGGAAGTTCACGCCTGCACGTTGGAACTTGGGATCTTTGCCGCCGTTGACAATCGGCGCGAGGAA |
| uNM_NM1 | AAGAGGGCGATATAGAGGAAGTTCACGCCTGCACGTTGGAACTTGGGATCTTTGCCGCCGTTGACAATCGGCGCGAGGAA |
| NM_LNP26948 | AAGAGGGCGATATAGAGGAAGTTTACGCCGGCACGTTGGAACTTGGGATCTTTACCGCCGTTGACAATCGGCGCGAGGAA |
| NM_FAM18 | AAGAGGGCGATATAGAGGAAGTTCACGCCTGCACGTTGGAACTTGGGATCTTTGCCGCCGTTGACAATCGGCGCAAGGAA |
| Nlac_020-06 | AACAGGGCGATATAGAGGAAGTTCACGCCTGCACGTTGGAACTTGGGATCTTTGCCGCCGTTGACAATCGGCGCGAGGAA |
| | ** ******************** ***** *********************** ******************** ***** |
| NG_NCCP11945 | CAAACCTGCCGTCAAAAAGCCGGTTGCAATCCAGAAGATGGCGGATTGGATGTGCCAAGTACGGGTCAGGGCGTAGGGGA |
| NG_FA1090 | CAAACCTGCCGTCAAAAAGCCGGTTGCAATCCAGAAGATGGCGGATTGGATGTGCCAAGTACGGGTCAGGGCGTAGGGGA |
| uNM_NM1 | CAAACCTGCCGTCAAAAAGCCGGTTGCAATCCAGAAGATGGCGGATTGGATGTGCCAAGTACGGGTCAGGGCGTAGGGGA |
| NM_LNP26948 | CAAACCTGCCGTCAAAAAGCCGGTTGCAATCCAGAAGATGGCTGATTGGATGTGCCAAGTACGGGTCAGGGCATAAGGGA |
| NM_FAM18 | CAAACCTGCCGTCAAAAAGCCGGTTGCAATCCAGAAGATGGCGGATTGGATGTGCCAAGTACGGGTCAGGGCGTAAGGGA |
| Nlac_020-06 | CAAACCTGCCGTCAAAAAGCCGGTTGCAATCCAGAAGATGGCGGATTGGATGTGCCAAGTACGGGTCAGGGCATAGGGGA |
| | ****************************************** ***************************** ** **** |
| NG_NCCP11945 | ACCAGTCGGACATTTCAAAGCCCAACGCCTCGTCAATGCCGTAGAAACCCTGACCTTCGACGGTGTAGTGCGCGGTCAGG |
| NG_FA1090 | ACCAGTCGGACATTTCAAAGCCCAACGCCTCGTCAATGCCGTAGAAACCCTGACCTTCGACGGTGTAGTGCGCGGTCAGG |
| uNM_NM1 | ACCAGTCGGACATTTCAAAGCCCAACGCCTCGTCAATGCCGTAGAAACCCTGACCTTCGACGGTGTAGTGCGCGGTCAGG |
| NM_LNP26948 | ACCAGTCGGACATTTCAAAGCCCAACGCTTCGTCGATGCCGTAGAAACCCTGGCCTTCGACGGTGTAGTGCGCGGTCAGT |
| NM_FAM18 | ACCAGTCGGACATTTCAAAGCCCAACGCCTCGTCAATGCCGTAGAAACCCTGACCTTCGACGGTGTAGTGCGCGGTCAGA |
| Nlac_020-06 | ACCAGTCGGACATTTCAAAGCCCAACGCCTCATCGATGCCGTAGAAACCCTGACCTTCGACGGTGTAGTGCGCGGTCAGA |
| | **************************** ** ** ***************** ************************** |
| NG_NCCP11945 | CCGCCCAGCAATACTTGTACCACAAACAGGGCGACCGTCAGGAAGACGTATTTGCCCAATGCTTTTTGCGAAGGGGTCAG |
| NG_FA1090 | CCGCCCAGCAATACTTGTACCACAAACAGGGCGACCGTCAGGAAGACGTATTTGCCCAATGCTTTTTGCGAAGGGGTCAG |
| uNM_NM1 | CCGCCCAGCAATACTTGTACCACAAACAGGGCGACCGTCAGGAAGACGTATTTGCCCAATGCTTTTTGCGAAGGGGTCAG |
| NM_LNP26948 | CCGCCCAGCAATACTTGTACCACAAACAGGGCGACCGTCAGGAAGACGTATTTGCCCAATGCTTTTTGCGAAGGGGTCAG |
| NM_FAM18 | CCGCCCAGCAATACTTGTACCACAAACAGGGCGACCGTCAGGAAGACGTATTTGCCCAATGCTTTTTGCGAAGGGGTCAG |
| Nlac_020-06 | CCGCCCAGCAATACTTGTACCACAAACAGGGCGACCGTCAGGAAGACGTATTTGCCCAATGCTTTTTGCGAAGGGGTCAG |
| | ******************************************************************************** |
| NG_NCCP11945 | TTGGATTTTGGAAATCGGGTCTTCAGACGGCACTTCCACTTCCTCGTGTTTGGTCAGGAAGGAATAACCCCACATCAACA |
| NG_FA1090 | TTGGATTTTGGAAATCGGGTCTTCAGACGGCACTTCCACTTCCTCGTGTTTGGTCAGGAAGGAATAACCCCACATCAACA |
| uNM_NM1 | TTGGATTTTGGAAATCGGGTCTTCAGACGGCACTTCCACTTCCTCGTGTTTGGTCAGGAAGGAATAACCCCACATCAACA |
| NM_LNP26948 | TTGGATTTTGGAAATCGGGTCTTCAGACGGCACTTCCACTTCCTCGTGTTTGGTCAAGAAGGAATAACCCCACATCAGCA |
| NM_FAM18 | TTGGATTTTGGAAATCGGGTCTTCAGACGGCACTTCCACTTCCTCGTGTTTGGTCAGGAAGGAATAACCCCACATCAACA |
| Nlac_020-06 | TTGGATTTTGGAAATCGGATCTTCAGCCGGGATTTCCACTTCCTCGTGTTTGGTCAGGAAGGAATAACCCCACATCAGCA |
| | ****************** ******* *** * *********************** ******************** ** |
| NG_NCCP11945 | AACCGATGCCCATCAAGAGCAGTACGACACTGGTAAACGACCACATGTAGTTTTCAGTAGTCGGTACATTGTTGATCAAA |
| NG_FA1090 | AACCGATGCCCATCAAGAGCAGTACGACACTGGTAAACGACCACATGTAGTTTTCAGTAGTCGGTACATTGTTGATCAAA |
| uNM_NM1 | AACCGATGCCCATCAAGAGCAGTACGACACTGGTAAACGACCACATGTAGTTTTCAGTAGTCGGTACATTGTTGATCAAA |
| NM_LNP26948 | AACCGATGCCCATCAGCAGAAGAACAACGCTGGTGAATGACCACATATAGTTTTCAGTGGTCGGTACGTTGTTGATCAAA |
| NM_FAM18 | AACCGATGCCCATCAGCAGAAGAACAACGCTGGTGAATGACCACATATAGTTTTCAGTGGTCGGTACGTTGTTGATCAAA |
| Nlac_020-06 | AACCGATGCCCATCAGCAGAAGAACAACGCTGGTAAACGACCACATATAGTTTTCAGTGGTCGGCACATTGTTGATCAAA |
| | *************** ** ** ** ** ***** ** ******** *********** ***** ** ************ |
| NG_NCCP11945 | GGCTCGTGCGGCCAGTTGTTGGTGTAGGTAAAGACCTCGCCGGGACGGTTGGTCGAAGCAGACCAAGAAGTCCAGAAGAA |
| NG_FA1090 | GGCTCGTGCGGCCAGTTGTTGGTGTAGGTAAAGACCTCGCCGGGACGGTTGGTCGAAGCAGACCAAGAAGTCCAGAAGAA |
| uNM_NM1 | GGCTCGTGCGGCCAGTTGTTGGTGTAGGTAAAGACCTCGCCGGGACGGTTGGTCGAAGCAGACCAAGAAGTCCAGAAGAA |
| NM_LNP26948 | GGCTCGTGCGGCCAGTTGTTGGTATAAGTGAACGTCTCGTCAGGACGGTTGGTCGAAGCAGACCAAGAAGTCCAGAAGAA |
| NM_FAM18 | GGCTCGTGCGGCCAGTTGTTGGTATAAGTGAACGTCTCGTCAGGACGGTTGGTCGAAGCAGACCAAGAAGTCCAGAAGAA |
| Nlac_020-06 | GGCTCGTGCGGCCAGTTGTTGGTGTAGGTAAAGACCTCGCCGGGACGGTTGGTCGAAGCAGACCAAGAAGTCCAAAAGAA |
| | *********************** ** ** ** **** * ******************************** ***** |
| NG_NCCP11945 | GAAGTCGAACAGTTTTTCACGCGCTTCTTGGCTTGGCAATGTGTTGTTTTTCATTGCAAAGTGTTCGCGGGTGGTTTGCA |
| NG_FA1090 | GAAGTCGAACAGTTTTTCACGCGCTTCTTGGCTTGGCAATGTGTTGTTTTTCATTGCAAAGTGTTCGCGGGTGGTTTGCA |
| uNM_NM1 | GAAGTCGAACAGTTTTTCACGCGCTTCTTGGCTTGGCAATGTGTTGTTTTTCATTGCAAAGTGTTCGCGGGTGGTTTGCA |
| NM_LNP26948 | GAAGTCGAACAGTTTTTCACGCGCTTCTTGGCTTGGCAATGTGTTGTTTTTCATTGCAAAGTGTTCGCGGGTGGTTTGCA |
| NM_FAM18 | GAAGTCGAACAGTTTTTCACGCGCTTCTTGGCTTGGCAATGTGTTGTTTTTCATTGCAAAGTGTTCGCGGGTGGTTTGCA |
| Nlac_020-06 | GAAGTCGAACAGTTTTTCACGCGCTTCTTGGCTTGGCAATGTGTTGTTTTTCATTGCAAAGTGTTCGCGGGTGGTTTGCA |
| | ******************************************************************************** |
| NG_NCCP11945 NG_FA1090 uNM_NM1 NM_LNP26948 NM_FAM18 Nlac_020-06 | 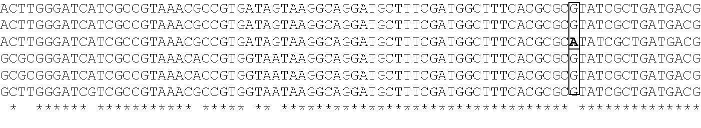 |
| NG_NCCP11945 | ACGCTGCCGTCTTCTTTAATACGGCTTTGATTGCGGTATTCATCGGCCAAGCGGGTTTTCAGAACGGCTTGTTCTTCAGG |
| NG_FA1090 | ACGCTGCCGTCTTCTTTAATACGGCTTTGATTGCGGTATTCATCGGCCAAGCGGGTTTTCAGAACGGCTTGTTCTTCAGG |
| uNM_NM1 | ACGCTGCCGTCTTCTTTAATACGGCTTTGATTGCGGTATTCATCGGCCAAGCGGGTTTTCAGAACGGCTTGTTCTTCAGG |
| NM_LNP26948 | ACGCTGCCGTCTTCTTTAATACGGCTTTGGTTGCGGTATTCGTCAGCCAGTCGGGTTTTCAGAACGGCTTGTTCTTCAGG |
| NM_FAM18 | ACGCTGCCGTCTTCTTTAATACGGCTTTGGTTGCGGTATTCGTCAGCCAGTCGGGTTTTCAGAACGGCTTGTTCTTCAGG |
| Nlac_020-06 | ACGCTGCCGTOTTCTTTCACACGGCTTTGGTTGCGGTATTCGTCAGCCAGGCGGGTTTTCAGAACGGCTTGTTCTTCAGG |
| | ***************** * ********* *********** ** **** **************************** |
| NG_NCCP11945 | GGAAACTTCATCGAATTTTTTGCCGTAAGCCTGTTGCGCGGTCAAATCCAACCAGGCGGACAACTCACGATGCAGCCAGT |
| NG_FA1090 | GGAAACTTCATCGAATTTTTTGCCGTAAGTCTGTTGCGCGGTCAAATCCAACCAGGCGGACAACTCACGATGCAGCCAGT |
| uNM_NM1 | GGAAACTTCATCGAATTTTTTGCCGTAAGCCTGTTGCGCGGTCAAATCCAACCAGGCGGACAACTCACGATGCAGCCAGT |
| NM_LNP26948 | GGAAACTTCATCGAATTTTTTGCCGTAAGTCTGTTGCGCGGTCAAATCCAACCAAGCGGACAACTCACGATGCAGCCAGT |
| NM_FAM18 | GGAAACTTCATCGAATTTTTTGCCGTAAGTCTGTTGCGCGGTCAAATCCAACTATGCAACCAACTCACGATGCAGCCAGT |
| Nlac_020-06 | GGAAACTTCATCAAATTTTTTGCCGTAAGTCTGTTGCGCGGTCAAATCCAACCAGGCAGACAACTCACGATGCAGCCAGT |
| | ************ **************** ********************** * ** ******************** |
| NG_NCCP11945 | CGGCCGTCCAATCCGGAGCCTGATATGCGCCGTGTCCCAGAATCGAACCGACTTCCATGCCGCCGGTAGTCTGCCACGCA |
| NG_FA1090 | CGGCCGTCCAGTCCGGAGCCTGATATGCGCCGTGACCCAGAATCGAACCGACTTCCATGCCGCCGGTACTCTGCCACGCA |
| uNM_NM1 | CGGCCGTCCAATCCGGAGCCTGATATGCGCCGTGTCCCAGAATCGAACCGACTTCCATGCCGCCGGTAGTCTGCCACGCA |
| NM_LNP26948 | CCGCCGTCCAGTCCGGAGCCTGATATGCGCCGTGACCCAGAATCGAACCGACTTCCATACCGCCGGTACTCTGCCACGCA |
| NM_FAM18 | CGGCCGTCCAGTCCGGAGCCTGATATGCGCCGTGACCCAGAATCGAACCGACTTCCATACCGCCGGTACTCTGCCACGCA |
| Nlac_020-06 | CGGCCGTCCAGTCCGGAGCCTGATATGCGCCGTGACCCAAAATCGAACCGACTTCCATACCGCCGGTACTCTGCCATGCA |
| | * ******** *********************** **** ****************** ********* ******* *** |
| NG_NCCP11945 | GACTGACCTGCCAAAATATCGTCTTTCGTCATCAGCACTTTGCCTGATGCGGAAACGACCTGTTCGGGGTAAGGCGGGGC |
| NG_FA1090 | GACTGACCTGCCAAAATATCGTCTTTCGTCATCAGCACTTTGCCGGATGCGGAAACGACCTGTTCGGGGTAAGGCGGGGC |
| uNM_NM1 | GACTGACCTGCCAAAATATCGTCTTTCGTCATCAGCACTTTGCCGGATGCGGAAACGACCTGTTCGGGGTAAGGCGGGGC |
| NM_LNP26948 | GACTGACCTGCCAAAATATCGTCTTTCGTCATCAGCACTTTGCCTGATGCGGAAACGACCTGTTCGGGGTAAGGCGGGGC |
| NM_FAM18 | GACTGACCTGCCAAAATATCGTCTTTCGTCATCAGCACTTTGCCTGATGCGGAAACGACCTGTTCAGGGTAAGGCGGGGC |
| Nlac_020-06 | GACTGACCTGCCAAAATATCGTOTTTCGTCATCAGCACTTTGCCTGATGCGGAAACGACCTGTTCGGGATAAGGCGGGGC |
| | ******************************************** ******************** ** *********** |
| NG_NCCP11945 | TTTCTTATAAACCTCGCTGCCCATATAGCCAAGAATGGTAAAGCATACCGCCAGAACGGCAAACAGCAAGTACCACAGCT |
| NG_FA1090 | TTTCTTATAAACCTCGCTGCCCATATAGCCAAGAATGGTAAAGCATACCGCCAGAACGGCAAACAGCAAGTACCACAGCT |
| uNM_NM1 | TTTCTTATAAACCTCGCTGCCCATATAGCCAAGAATGGTAAAGCATACCGCCAGAACGGCAAACAGCAAGTACCACAGCT |
| NM_LNP26948 | TTTCTTATAAACCTCGCTGCCCATATAGCCAAGAATGGTAAAGCATACCGCCAGAACGGCAAACAGCAAGTACCACAGCT |
| NM_FAM18 | TTTTTTGTAAACCTCGCTGCCCATATAGCCAAGAATGGTAAAGCATACCGCCAGAACGGCAAACAGCAAGTACCATAGCT |
| Nlac_020-06 | TTTCTTATAAACCTCGCTGCCCATATAGCCGAGAATGGTAAAGCATACCGCCAGAACGGCGAACAGCAAGTACCACAGCT |
| | *** ** *********************** ***************************** ************** **** |
| NG_NCCP11945 | TCTTGTACTGTCCCAT |
| NG_FA1090 | TCTTGTACTGTCCCAT |
| uNM_NM1 | TCTTGTACTGTCCCAT |
| NM_LNP26948 | TCTTGTACTGTCCCAT |
| NM_FAM18 | TCTTGTACTGTCCCAT |
| Nlac_020-06 | TCTTATACTGTCCCAT |
| | **** *********** |

### Example 8

### Cross-Reaction with Other Urogenital Microorganisms

A variety of pathogenic and commensal microorganisms commonly colonize the distal male urethra, so it was determined if high loads of DNA from common urethral microorganisms interfered with performance of the assay. Urine matrices were spiked with various organisms including CT, TV, MG, N. cinerea, N. lactamica, N. perflava, N. subflava; genomic DNA from Ureaplasma urealyticum, HSV 1, HSV2, HPV 16; or the control plasmids pNM1-norB and pNG-norB, and total DNA from the mixtures was extracted and used as template in the Simpleprobe assay. Contrived specimens spiked with the two uNM isolates (NM1 and NM2), and the uNM plasmid control plasmid yielded a melting peak around 57° C., whereas none of the contrived specimens spiked with other Neisseria species or our select panel of urogenital microorganisms yielded a melting peak near this temperature. Contrived specimens spiked with NG, N. lactamica and the controls containing NG, or the NG plasmid yielded a melting peak around 66° C., whereas none of the other microorganisms amplified. Taken together, the two clearly separated melting peaks showed complete predictive concordance with NG and uNM ST-11 strain calls. (FIG. 7).

### Example 9

### The uNM ST-11 Simpleprobe Assay is Insensitive to High Concentrations of NG norB

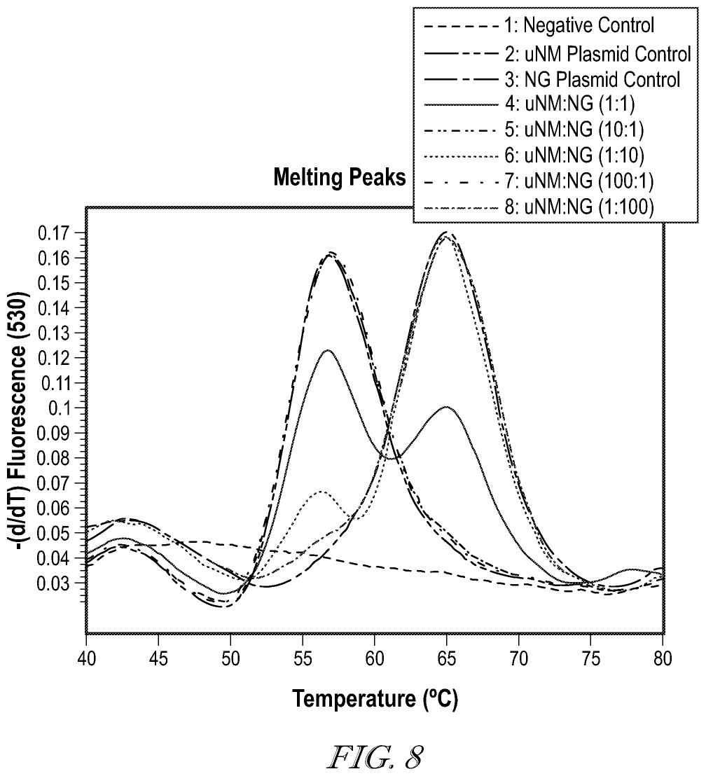

The assay had a low LOD and yielded distinct melting peaks for NM1 and NG when these organisms were present singly in relevant matrices. Since men with urethritis commonly have multiple STI, the effects of different ratios of pNG-norB and pNM1-norB on the assay performance were determined (FIG. 8). The amplitudes of the NM1 and NG amplification peaks reflected the ratios of the corresponding templates in the reaction mixtures, and the uNM ST-11 and NG norB melting peaks were distinct at ratios ranging from 1:100 to 100:1 of pNG-norB and pNM1-norB. This result confirmed that the uNM ST-11 Simpleprobe assay can differentiate uNM ST-11 in NG co-infected specimens.

### Example 10

### Validation of the uNM Simpleprobe Assay in Clinical Specimens

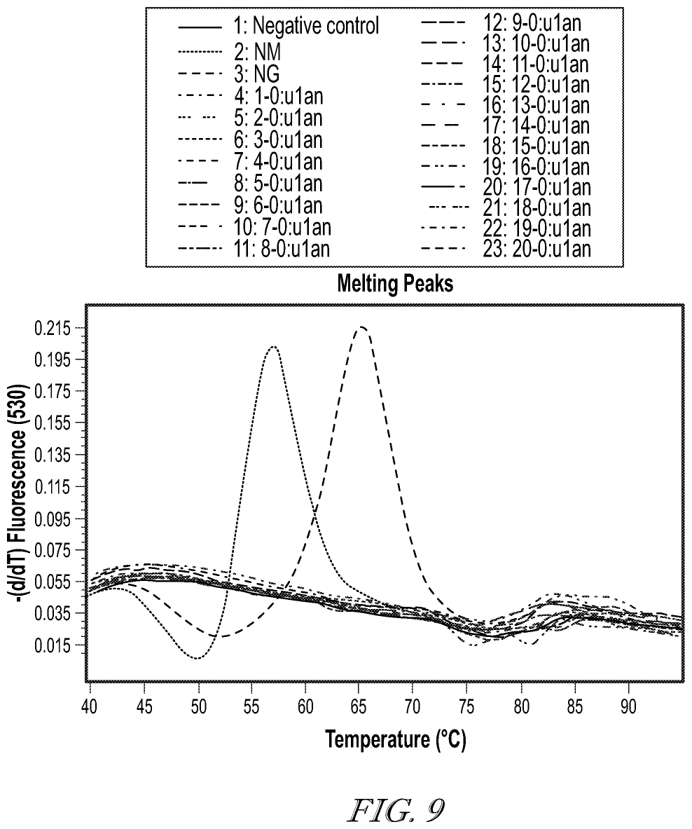

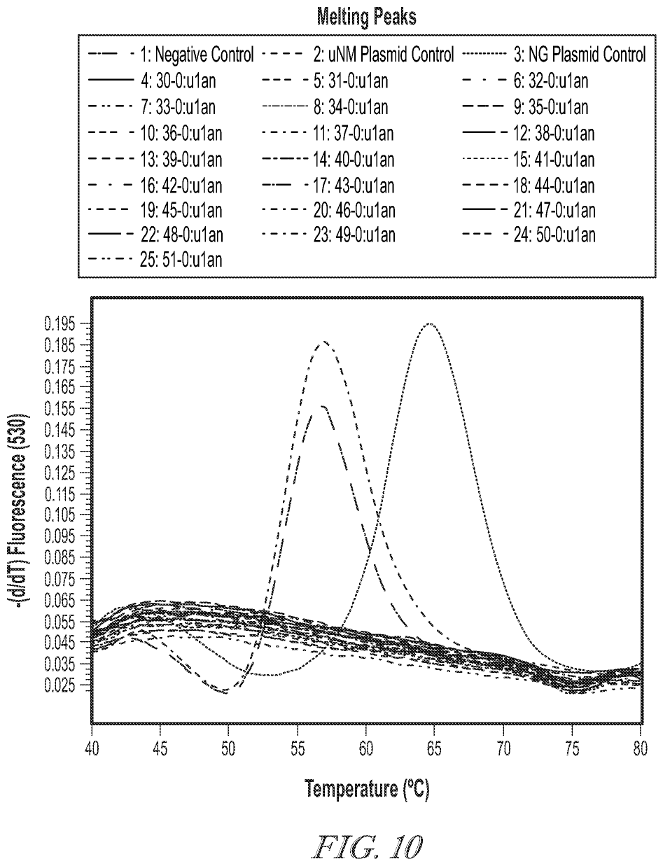

Archived specimens from an ongoing study were used to validate the assay using clinical specimens. Urines were available from 241 men (enrolled between Aug. 4, 2016 to Jul. 13, 2018), including 127 NGU cases and 114 healthy controls which tested NG negative by NAAT. Deep shotgun metagenomic sequencing of urethral swab specimens from the same men, sequenced to an average depth of 7 gigabases, identified one case (Case 43) whose specimen contained a high proportion of NM sequences. Analysis of the same specimen collection using a Taqman PCR targeting Neisserial metA (31) identified three positive specimens, case 43, case 110, and healthy control 1062. PCR amplification and sequencing of norB from the swabs and urines from case 43 and case 110 confirmed that case 43 was colonized with uNM ST-11, whereas case 110 and control 1062 were colonized with commensal NM strains. When the entire collection of urines was analyzed with the uNM ST-11 assay by a blinded technician, case 43 also yielded the expected amplification curve for uNM ST-11, and the NG plasmid control yielded the expected melting peak for NG; whereas all of the other specimens yielded no amplification. To ensure that the lack of metA positive samples was not due to a lack of sensitivity of this assay, we performed the SimpleProbe assay on the first 20 IUMP samples and on a subsequent 21 samples that contained the one sample that was positive for metA. None of the samples that were metA negative were positive by the SimpleProbe assay. (FIG. 9 and FIG. 10).

A simple and sensitive assay for the detection of the urethrotropic NM clade strains that does not require microbial culture or genome sequencing has been developed. Performance of the assay is robust with male clinical urine and urethral specimens and contrived vaginal specimens. The assay may easily be adapted for use with other clinical specimen types and high throughput testing platforms present in most contemporary STI diagnostic laboratories. This would facilitate epidemiological studies and could define how the NM urethritis strains are transmitted and their pathogenic potential.

### Example 11

### Endpoint Genotyping Assay for Detection of uNM

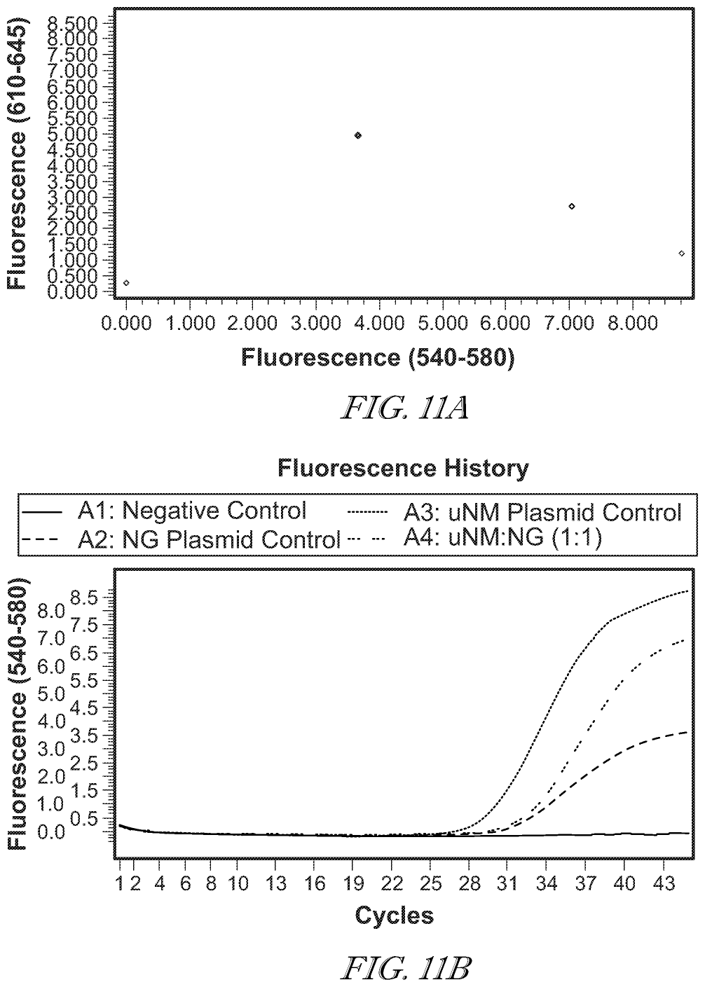

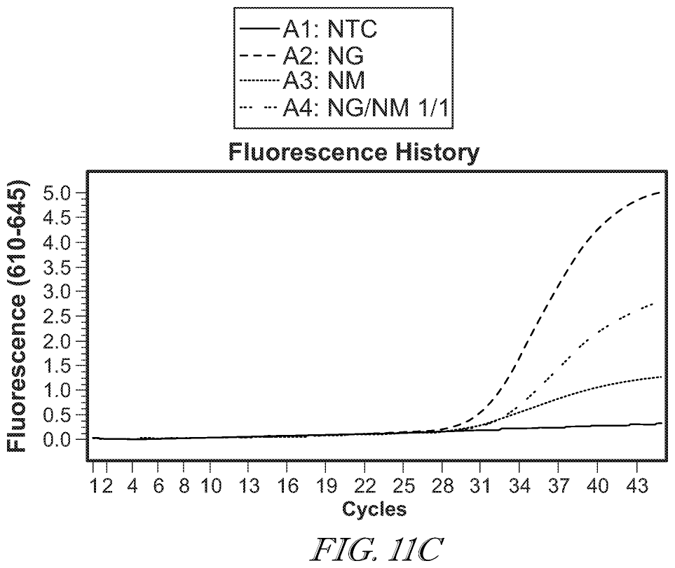

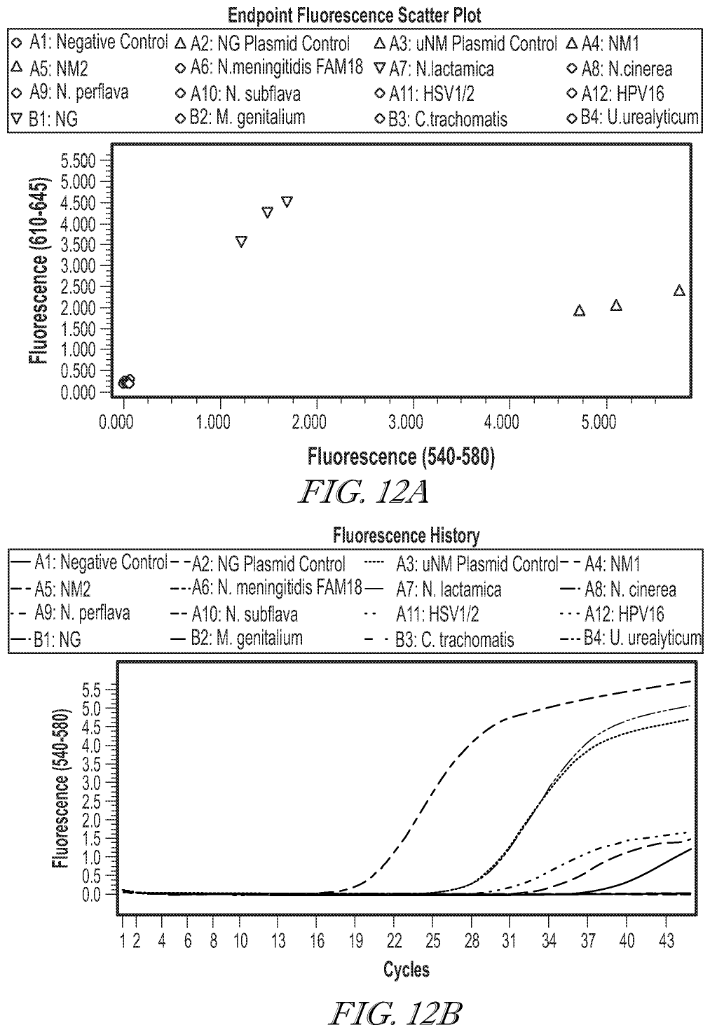

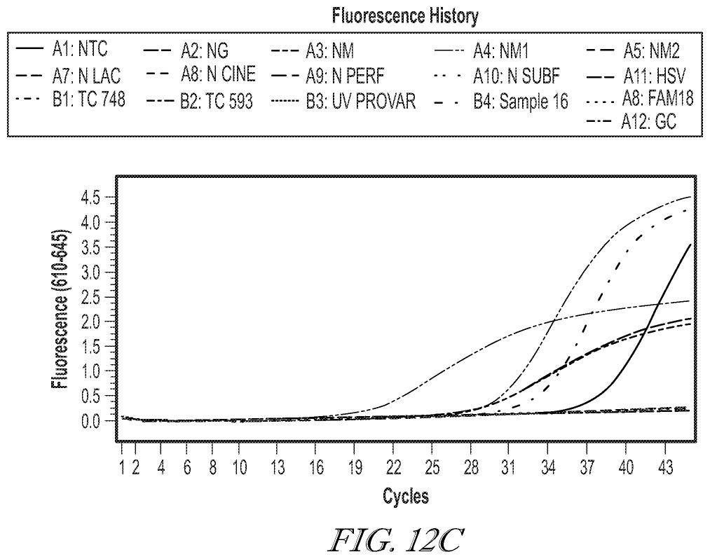

Two differently labelled hydrolysis probes were designed that bind either to “A” (NG, wild type) or “G” (uNM, mutant) at nucleotide position 431 of the norB gene. The primers and fluorescently labeled probes for this assay are listed in Table 10 below. Reactions were set-up using the LightCycler480 Probe Master Mix in a total volume of 20 μl. The final concentrations of uNM forward and reverse primers were 0.2 uM, and 0.3 uM respectively. The P-NG-WT431A and P-NM-MT431G probe concentrations were 0.4 uM, and 0.3 uM respectively. The multiplex assay PCR profile were as follows: 10 min of 95° C. (ramp rate 4.4° C./s), followed by 45 cycles of 95° C. for 10 seconds (ramp rate 4.4° C./s), and 61° C. for 25 seconds (single acquisition mode and ramp rate of 2.2° C./s) (Table 11). All amplification reactions were performed using the Roche LightCycler z480 instrument (Roche Diagnostics, Indianapolis, Ind.). Generation of a fluorescent signal was dependent upon whether the target DNA sequence in the sample is a match for either the wild type or mutant probe or both in the case of a mixed infection. Fluorescent intensity is measured on the z480 using the 540-580 nm channel for uNM (HEX) and the 610-640 nm channel for NG (TEX615). The ratios of fluorescence intensities can be used to determine which allele the hydrolysis probes bound to. These ratios were used to identify whether the samples are positive for the presence of uNM, NG, or a mixture of both. In addition, a collection of anogenital pathogens and other Neisserial species obtained from the American Type Culture Collection (ATCC) and other sources were used to determine the sensitivity and cross reactivity of this end point genotyping assay. Results are shown in FIG. 11 (panels A, B, and C) and FIG. 12 (panels A, B, and C). Results are also shown below in Tables 12 and 13.

TABLE 10
1-NM and NG Endpoint genotyping primers and probes

| Primer/Probe Name | Primer/Probe Sequence |
|---|---|
| Forward | GCTTGGCCGATGAATACC |
| Reverse | CGTAAACGCCGTGATAGT |
| Wild type Probe: | TEX615/GCGATACGCGCGTGAAAGCCAT/ |
| P-NG-WT431A | 3IAbQ |
| Mutant Probe: | HEX/CGATATGCGCGTGAAAGCCAT/BHQ1 |
| P-NM-MT431G | |

TABLE 11

| | Programs |
|---|---|
| Program Name | Program |

| Cycles | 1 | Analysis Mode | None | | | | |
|---|---|---|---|---|---|---|---|
| Target | Acquisition | Hold | Ramp Rate | Acquisitions | Sec Target | Step size | Step Delay |
| (° C.) | Mode | (hr:mm:ss) | (° C./s) | (per ° C.) | (° C.) | (° C.) | (cycles) |
| 95 | None | 00:10:00 | 4.40 | | 0 | 0 | 0 |
| Program Name | Program | | | | | | |
| Cycles | 45 | Analysis Mode | Quantification | | | | |
| Target | Acquisition | Hold | Ramp Rate | Acquisitions | Sec Target | Step size | Step Delay |
| (° C.) | Mode | (hr:mm:ss) | (° C./s) | (per ° C.) | (° C.) | (° C.) | (cycles) |
| 95 | None | 00:00:10 | 4.40 | | 0 | 0 | 0 |
| 61 | Single | 00:00:25 | 2.20 | | 0 | 0 | 0 |

TABLE 12
Results Endpoint Genotyping for New Subset 1 (FIGS. 11 A, B, and C).

| | | | Endpoint Fluorescence | | | | |
|---|---|---|---|---|---|---|---|
| Inc. | Pos. | Sample Name | Allele X | Allele Y | Call | Score | Status |
| | A1 | Negative Control | 0.00 | 0.27 | Negative | | |
| | A2 | NG Plasmid Control | 3.65 | 4.99 | WT | 0.67 | |
| | A3 | uNM Plasmid Control | 8.77 | 1.24 | MT | 0.66 | |
| | A4 | uNM:NG (1:1) | 7.03 | 2.74 | MT | 0.91 | |

TABLE 13
Results Endpoint Genotyping for New Subset 1 (FIGS. 12 A, B, and C).
Results

| | | | Endpoint Fluorescence | | | | |
|---|---|---|---|---|---|---|---|
| Inc. | Pos. | Sample Name | Allele X | Allele Y | Call | Score | Status |
| | A1 | Negative Control | 0.06 | 0.28 | Negative | | |
| | A2 | NG Plasmid Control | 1.49 | 4.27 | WT | 0.93 | |
| | A3 | uNM Plasmid Control | 4.72 | 1.94 | MT | 0.92 | |
| | A4 | NM1 | 5.75 | 2.42 | MT | 0.94 | |
| | A5 | NM2 | 5.10 | 2.05 | MT | 0.92 | |
| | A6 | N. meningitis FAM18 | 0.06 | 0.24 | Negative | | |
| | A7 | N. lacternica | 1.69 | 4.52 | WT | 0.96 | |
| | A8 | N. cinerea | 0.02 | 0.19 | Negative | | |
| | A9 | N. perflava | 0.00 | 0.20 | Negative | | |
| | A10 | N. subflava | 0.00 | 0.19 | Negative | | |
| | A11 | HPV1/2 | 0.01 | 0.26 | Negative | | |
| | A12 | HPV18 | 0.07 | 0.29 | Negative | | |
| | B1 | NG | 1.21 | 3.55 | WT | 0.93 | |
| | B2 | M. genitalium | 0.04 | 0.25 | Negative | | |
| | B3 | C. trachomatis | 0.05 | 0.26 | Negative | | |
| | B4 | U. urealyticum | 0.04 | 0.23 | Negative | | |

1. A method of preparing a DNA fraction from a patient's sample useful for detecting a urethrotropic strain of Neisseria meningitidis comprising:
extracting and recovering bacterial DNA from a patient sample;
selectively amplifying the bacterial DNA using a forward and reverse primer to produce an amplicon;
contacting said amplified bacterial DNA with a labeled nucleic acid probe, wherein said labeled probe comprises a nucleic acid selected from the group consisting of 5′-CGTCATCAGCGATACGCG-3′(SEQ ID NO: 1), 5′-GCGATACGCGCGTGAAAGCCAT-3′ (SEQ ID NO: 2), and 5′-CGATATGCGCGTGAAAGCCAT-3′(SEQ ID NO: 3) or a nucleic acid having at least 95% sequence identity to the nucleic acid of SEQ ID NO: 1, SEQ ID NO: 2 or SEQ ID NO: 3, or a complement of any of said nucleic acids; and
identifying a urethrotropic strain of Neisseria meningitidis in the patient sample, by detecting hybridization between said amplicon and said labeled probe.

2. The method of claim 1, wherein the DNA is amplified using PCR.

3. The method of claim 2, wherein the PCR is an asymmetric PCR.

4. The method of claim 2, wherein the PCR is real-time PCR.

5. The method of claim 1, wherein the probe is fluorescently labeled.

6. (canceled)

7. The method of claim 2, wherein the forward primer is selected from the group consisting of 5′-GCTTGGCCGATGAATACC-3′ (SEQ ID NO: 4) and 5′-GCTGATGACGAAAGACGA-3′ (SEQ ID NO: 5).

8. The method of claim 2, wherein the reverse primer is selected from the group consisting of 5′-CAATGAAAAACAACACAT-3′ (SEQ ID NO: 6) and 5′-CGTAAACGCCGTGATAGT-3′ (SEQ ID NO: 7).

9. The method of claim 1, wherein the difference in melting temperatures (Tm) between the products of the PCR reaction and the fluorescence produced during a melting curve analysis is determined.

10. The method of claim 1, further comprising quantifying the amount of urethrotropic Neisseria meningitidis (uNM) in the sample.

11. A method of preparing a DNA fraction from a patient's sample useful for detecting a urethrotropic strain of Neisseria meningitidis said method comprising:
extracting and recovering bacterial DNA from a patient sample;
selectively amplifying the bacterial DNA using a forward and reverse primer to produce an amplicon;
contacting said amplified bacterial DNA with a labeled nucleic acid probe, wherein said probe is specific for a gene from Neisseria gonorrhoeae (NG); and
identifying a urethrotropic strain of Neisseria meningitidis in the patient sample, by detecting hybridization between said amplicon and said labeled probe.

12. The method of claim 11, wherein said Neisseria gonorrhoeae gene is the norB gene.

13. The method of claim 12, wherein the probe targets a single nucleotide polymorphism in the norB gene.

14. The method of claim 9, wherein the difference in melting temperature of the products detects a single nucleotide polymorphism in the norB gene.

15. The method of claim 1, wherein the patient sample is a patient body fluid selected from the group consisting of urine, seminal fluid, vaginal fluid, other reproductive tract secretions, lymph fluid, whole blood, serum, and plasma.

16. The method of claim 1, wherein the patient has urethritis.

17. The method of claim 1, wherein the strain of Neisseria meningitidis is not a commensal strain.

18. (canceled)

19. A kit comprising a purified nucleic acid comprising a sequence selected from 5′-GCTGATGACGAAAGACGA-3′(SEQ ID NO: 5), 5′-GCTTGGCCGATGAATACC-3′(SEQ ID NO: 4), 5′-CAATGAAAAACAACACAT-3′(SEQ ID NO: 6) and 5′-CGTAAACGCCGTGATAGT-3′(SEQ ID NO: 7), and a fluorescently labeled probe.

20. The kit of claim 19, wherein the fluorescently labeled probe comprises a DNA sequence selected from the group consisting of 5′-CGTCATCAGCGATACGCG-3′(SEQ ID NO: 1), 5′-GCGATACGCGCGTGAAAGCCAT-3′(SEQ ID NO: 2), and 5′-CGATATGCGCGTGAAAGCCAT-3′(SEQ ID NO: 3).


SEQUENCE LISTING (16 sequences)

SEQ ID NO: 1 - DNA, 18 bp, Artificial Sequence
  Description of Artificial Sequence Synthetic oligonucleotide
cgtcatcagcgatacgcg

SEQ ID NO: 2 - DNA, 22 bp, Artificial Sequence
  Description of Artificial Sequence Synthetic oligonucleotide
gcgatacgcgcgtgaaagccat

SEQ ID NO: 3 - DNA, 21 bp, Artificial Sequence
  Description of Artificial Sequence Synthetic oligonucleotide
cgatatgcgcgtgaaagccat

SEQ ID NO: 4 - DNA, 18 bp, Artificial Sequence
  Description of Artificial Sequence Synthetic oligonucleotide
gcttggccgatgaatacc

SEQ ID NO: 5 - DNA, 18 bp, Artificial Sequence
  Description of Artificial Sequence Synthetic oligonucleotide
gctgatgacgaaagacga

SEQ ID NO: 6 - DNA, 18 bp, Artificial Sequence
  Description of Artificial Sequence Synthetic oligonucleotide
caatgaaaaacaacacat

SEQ ID NO: 7 - DNA, 18 bp, Artificial Sequence
  Description of Artificial Sequence Synthetic oligonucleotide
cgtaaacgccgtgatagt

SEQ ID NO: 8 - DNA, 18 bp, Artificial Sequence
  Description of Artificial Sequence Synthetic oligonucleotide
  5′-Fluorescein-SPC
  3′-Phosphate
cgtcatcagcgatacgcg

SEQ ID NO: 9 - DNA, 22 bp, Artificial Sequence
  Description of Artificial Sequence Synthetic oligonucleotide
  5′-TEX615
  3′-3IAbQ
gcgatacgcgcgtgaaagccat

SEQ ID NO: 10 - DNA, 21 bp, Artificial Sequence
  Description of Artificial Sequence Synthetic oligonucleotide
  5′-HEX
  3′-BHQ1
cgatatgcgcgtgaaagccat

SEQ ID NO: 11 - DNA, 2256 bp, Neisseria gonorrhoeae
ttatttgtcgcggccgaatacgattttagtggcttggatggcaacgcagattgcaccgccgataaagattaagtcgggggcagtgcgtacccagcgcagggtgtcgaggatttccatttgcaggaattcttcgctgcgggcataccacaaaccgtgcgtgatagaggcgtatgcctgaatcacgccgacaggcagcaggctgatggcaatcataccgaccaagccgccgttgagcagccagaagccccaagtcatcagtttgtcgtcaaaccgcgcgttcggtttcaagtagcgcgcaaccaacaatacgaagcccaatgccaagaaaccgtacacaccgaacaaggcggcgtgcgcgtgaacggcggaagtgttcaaaccttggatatagaacagggaaatcggcggattgatcagaaagccgaatacgccggcaccgatcatattccaaaaagcgactgccacgaagcacatcagcggccaacgcaggcgtttcgcccagtcggacaggtgttggtaagaccagtgctcgtatgcctcgcggcccagcaacaccagcggcacgacttccaaagcggagaagcaggcgccgattgccatagaggcggaggtagagccggagaagtacaggtggtgcagcgtgcccggaacgccgcccaacataaagatggcggcagcggccaaagtggaggcagtggcggtactgcggcggacaaagcccatattgtagaagacaaaggcaaaggcggcagtggcaaatacttcaaagaagccttccacccacaggtggaccacccaccaacgccagtattccattacggcaatcggggatttttcgccatagaacaggcccggtgcgtagaacacgcccacgccgaccatagaagcgacaaagattgccagcaggtttttgtccacgcctttttctttgaaggcggaaaccgtgcagcgcaacatcaggaacagccacaacagcagtccgaccatcaaaaggagttgccagaaacgtcccaaatcgaggtattcgtaaccttggtgtccgaaccagaagttaaattcggggggaaggatgtgcgtcaacgcgaagaagttgcccgcgtaagaaccgccgaccacgatgaagagggcgatatagaggaagttcacgcctgcacgttggaacttgggatctttgccgccgttgacaatcggcgcgaggaacaaacctgccgtcaaaaagccggttgcaatccagaagatggcggattggatgtgccaagtacgggtcagggcgtaggggaaccagtcggacatttcaaagcccaacgcctcgtcaatgccgtagaaaccctgaccttcgacggtgtagtgcgcggtcaggccgcccagcaatacttgtaccacaaacagggcgaccgtcaggaagacgtatttgcccaatgctttttgcgaaggggtcagttggattttggaaatcgggtcttcagacggcacttccacttcctcgtgtttggtcaggaaggaataaccccacatcaacaaaccgatgcccatcaagagcagtacgacactggtaaacgaccacatgtagttttcagtagtcggtacattgttgatcaaaggctcgtgcggccagttgttggtgtaggtaaagacctcgccgggacggttggtcgaagcagaccaagaagtccagaagaagaagtcgaacagtttttcacgcgcttcttggcttggcaatgtgttgtttttcattgcaaagtgttcgcgggtggtttgcaacttgggatcatcgccgtaaacgccgtgatagtaaggcaggatgctttcgatggctttcacgcgcgtatcgctgatgacgacgctgccgtcttctttaatacggctttgattgcggtattcatcggccaagcgggttttcagaacggcttgttcttcaggggaaacttcatcgaattttttgccgtaagcctgttgcgcggtcaaatccaaccaggcggacaactcacgatgcagccagtcggccgtccaatccggagcctgatatgcgccgtgtcccagaatcgaaccgacttccatgccgccggtagtctgccacgcagactgacctgccaaaatatcgtctttcgtcatcagcactttgcctgatgcggaaacgacctgttcggggtaaggcggggctttcttataaacctcgctgcccatatagccaagaatggtaaagcataccgccagaacggcaaacagcaagtaccacagcttcttgtactgtcccat

SEQ ID NO: 12 - DNA, 2256 bp, Neisseria gonorrhoeae
ttatttgtcgcggccgaatacgattttagtggcttggatggcaacgcagattgcaccgccgataaagattaagtcgggggcagtgcgtacccagcgcagggtgtcgaggatttccatttgcaggaattcttcgctgcgggcataccacaaaccgtgcgtgatagaggcgtatgcctgaatcacgccgacaggcagcaggctgatggcaatcataccgaccaagccgccgttgagcagccagaagccccaagtcatcagtttgtcgtcaaaccgcgcgttcggtttcaagtagcgcgcaaccaacaatacgaagcccaatgccaagaaaccgtacacaccgaacaaggcggcgtgcgcgtgaacggcggaagtgttcaaaccttggatatagaacagggaaatcggcggattgatcagaaagccgaatacgccggcaccgatcatattccaaaaagcgactgccacgaagcacatcagcggccaacgcaggcgtttcgcccagtcggacaggtgttggtaagaccagtgctcgtatgcctcgcggcccagcaacaccagcggcacgacttccaaagcggagaagcaggcgccgattgccatagaggcggaggtagagccggagaagtacaggtggtgcagcgtgcccggaacgccgcccaacataaagatggcggcagcggccaaagtggaggcagtggcggtactgcggcggacaaagcccatattgtagaagacaaaggcaaaggcggcagtggcaaatacttcaaagaagccttccacccacaggtggaccacccaccaacgccagtattccattacggcaatcggggatttttcgccatagaacaggcccggtgcgtagaacacgcccacgccgaccatagaagcgacaaagattgccagcaagtttttgtccacgcctttttctttgaaggcggaaaccgtgcagcgcaacatcaggaacagccacaacagcagtccgaccatcaaaaggagttgccagaaacgtcccaaatcgaggtattcgtaaccttggtgtccgaaccagaagttaaattcgggaggaaggatgtgcgtcaacgcgaagaagttgcccgcgtaagaaccgccgaccacgatgaagagggcgatatagaggaagttcacgcctgcacgttggaacttgggatctttgccgccgttgacaatcggcgcgaggaacaaacctgccgtcaaaaagccggttgcaatccagaagatggcggattggatgtgccaagtacgggtcagggcgtaggggaaccagtcggacatttcaaagcccaacgcctcgtcaatgccgtagaaaccctgaccttcgacggtgtagtgcgcggtcaggccgcccagcaatacttgtaccacaaacagggcgaccgtcaggaagacgtatttgcccaatgctttttgcgaaggggtcagttggattttggaaatcgggtcttcagacggcacttccacttcctcgtgtttggtcaggaaggaataaccccacatcaacaaaccgatgcccatcaagagcagtacgacactggtaaacgaccacatgtagttttcagtagtcggtacattgttgatcaaaggctcgtgcggccagttgttggtgtaggtaaagacctcgccgggacggttggtcgaagcagaccaagaagtccagaagaagaagtcgaacagtttttcacgcgcttcttggcttggcaatgtgttgtttttcattgcaaagtgttcgcgggtggtttgcaacttgggatcatcgccgtaaacgccgtgatagtaaggcaggatgctttcgatggctttcacgcgcgtatcgctgatgacgacgctgccgtcttctttaatacggctttgattgcggtattcatcggccaagcgggttttcagaacggcttgttcttcaggggaaacttcatcgaattttttgccgtaagtctgttgcgcggtcaaatccaaccaggcggacaactcacgatgcagccagtcggccgtccagtccggagcctgatatgcgccgtgacccagaatcgaaccgacttccatgccgccggtactctgccacgcagactgacctgccaaaatatcgtctttcgtcatcagcactttgccggatgcggaaacgacctgttcggggtaaggcggggctttcttataaacctcgctgcccatatagccaagaatggtaaagcataccgccagaacggcaaacagcaagtaccacagcttcttgtactgtcccat

SEQ ID NO: 13 - DNA, 2256 bp, Neisseria meningitidis
ttatttgtcgcggccgaatacgattttagtggcttggatggcaacgcagattgcaccgccgataaagattaagtcgggggcagtgcgtacccagcgcagggtgtcgaggatttccatttgcaggaattcttcgctgcgggcataccacaaaccgtgcgtgatagaggcgtatgcctgaatcacgccgacaggcagcaggctgatggcaatcataccgaccaagccgccgttgagcagccagaagccccaagtcatcagtttgtcgtcaaaccgcgcgttcggtttcaagtagcgcgcaaccaacaatacgaagcccaatgccaagaaaccgtacacaccgaacaaggcggcgtgcgcgtgaacggcggaagtgttcaaaccttggatatagaacagggaaatcggcggattgatcagaaagccgaatacgccggcaccgatcatattccaaaaagcgactgccacgaagcacattagcggccaacgcaggcgtttcgcccagtcggacaggtgttggtaagaccagtgctcgtatgcctcgcggcccagcaacaccagcggcacgacttccaaagcggagaagcaggcgccgattgccatagaggcggaggtagagccggagaagtacaggtggtgcagcgtgcccggaacgccgcccaacataaagatggcggcagcggccaaagtggaggcagtggcggtactgcggcggacaaagcccatattgtagaagacaaaggcaaaggcggcagtggcaaatacttcaaagaagccttccacccacaggtggaccacccaccaacgccagtattccattacggcaatcggggatttttcgccatagaacaggcccggtgcgtagaacacgcccacgccgaccatagaagcgacaaagattgccagcaagtttttgtccacgcctttttctttgaaggcggaaaccgtgcagcgcaacatcaggaacagccacaacagcagtccgaccatcaaaaggagttgccagaaacgtcccaaatcgaggtattcgtaaccttggtgtccgaaccagaagttaaattcgggaggaaggatgtgcgtcaacgcgaagaagttgcccgcgtaagaaccgccgaccacgatgaagagggcgatatagaggaagttcacgcctgcacgttggaacttgggatctttgccgccgttgacaatcggcgcgaggaacaaacctgccgtcaaaaagccggttgcaatccagaagatggcggattggatgtgccaagtacgggtcagggcgtaggggaaccagtcggacatttcaaagcccaacgcctcgtcaatgccgtagaaaccctgaccttcgacggtgtagtgcgcggtcaggccgcccagcaatacttgtaccacaaacagggcgaccgtcaggaagacgtatttgcccaatgctttttgcgaaggggtcagttggattttggaaatcgggtcttcagacggcacttccacttcctcgtgtttggtcaggaaggaataaccccacatcaacaaaccgatgcccatcaagagcagtacgacactggtaaacgaccacatgtagttttcagtagtcggtacattgttgatcaaaggctcgtgcggccagttgttggtgtaggtaaagacctcgccgggacggttggtcgaagcagaccaagaagtccagaagaagaagtcgaacagtttttcacgcgcttcttggcttggcaatgtgttgtttttcattgcaaagtgttcgcgggtggtttgcaacttgggatcatcgccgtaaacgccgtgatagtaaggcaggatgctttcgatggctttcacgcgcatatcgctgatgacgacgctgccgtcttctttaatacggctttgattgcggtattcatcggccaagcgggttttcagaacggcttgttcttcaggggaaacttcatcgaattttttgccgtaagcctgttgcgcggtcaaatccaaccaggcggacaactcacgatgcagccagtcggccgtccaatccggagcctgatatgcgccgtgtcccagaatcgaaccgacttccatgccgccggtagtctgccacgcagactgacctgccaaaatatcgtctttcgtcatcagcactttgccggatgcggaaacgacctgttcggggtaaggcggggctttcttataaacctcgctgcccatatagccaagaatggtaaagcataccgccagaacggcaaacagcaagtaccacagcttcttgtactgtcccat

SEQ ID NO: 14 - DNA, 2256 bp, Neisseria meningitidis
ttatttgtcgcggccgaatacgattttagtggcttggatggcaacgcagattgcaccgccgataaagaccaagtcagctgccgtacgtacccaacgcaaggtgtcgaggatttccatttgcaggaactcttcgctgcgtgcataccacaggcggtgcgtgatggaggcgtatgcctgaatcacgccaaccggcaacaggctgatggcaatcataccgaccaagccgccgttgagcagccagaagccccaagtcatcagtttgtcgtcaaactgcgcgttcggtttcaaataacgggcaaccagcaatacgaagcccaatgccaagaaaccgtacacaccgaacaaggcggcgtgcgcatgaacagcagaagtattcaaaccttggatatagaacagggaaatcggcgggttaatcaggaaaccgaatacaccggcaccgatcatattccaaaaggcgactgccacgaagcacatcagcggccaacgcaggcgtttcgcccagtcggacaggtgttggtaagaccaatgctcgtatgcttcacggcccagcaacaccagcggcacgacttccaaagcggagaagcaggcaccgattgccatagaggcggaggtagagccggagaagtacaggtggtgcagcgtacccggaacgccgcccaacataaagatggcggcagcggccaaagtagaagctgtggcggtactgcggcggacaaagcccatattgtagaagacaaatgcgaaggcggcagtggcaaatacttcaaagaagccttccacccacaggtgaaccacccaccaacgccagtattccattacggcaatcggggatttctcgccatagaacagtcccggtgcgtagaatacgcccacaccgaccatagaagcgacaaagattgccagcaggtttttgtccacgcctttttctttgaaggcggaaaccgtgcaacgcaacatcaggaacagccacaacagcaggccgaccatcagcaggagttgccagaaacgtcccaaatcgaggtattcgtaaccttggtgtccgaaccagaagttaaattccgggggaaggatgtgcgtcaacgcgaagaagttgcccgcgtaagaaccgccgaccacgatgaagagggcgatatagaggaagtttacgccggcacgttggaacttgggatctttaccgccgttgacaatcggcgcgaggaacaaacctgccgtcaaaaagccggttgcaatccagaagatggctgattggatgtgccaagtacgggtcagggcataagggaaccagtcggacatttcaaagcccaacgcttcgtcgatgccgtagaaaccctggccttcgacggtgtagtgcgcggtcagtccgcccagcaatacttgtaccacaaacagggcgaccgtcaggaagacgtatttgcccaatgctttttgcgaaggggtcagttggattttggaaatcgggtcttcagacggcacttccacttcctcgtgtttggtcaagaaggaataaccccacatcagcaaaccgatgcccatcagcagaagaacaacgctggtgaatgaccacatatagttttcagtggtcggtacgttgttgatcaaaggctcgtgcggccagttgttggtataagtgaacgtctcgtcaggacggttggtcgaagcagaccaagaagtccagaagaagaagtcgaacagtttttcacgcgcttcttggcttggcaatgtgttgtttttcattgcaaagtgttcgcgggtggtttgcagcgcgggatcatcgccgtaaacaccgtggtaataaggcaggatgctttcgatggctttcacgcgcgtatcgctgatgacgacgctgccgtcttctttaatacggctttggttgcggtattcgtcagccagtcgggttttcagaacggcttgttcttcaggggaaacttcatcgaattttttgccgtaagtctgttgcgcggtcaaatccaaccaagcggacaactcacgatgcagccagtccgccgtccagtccggagcctgatatgcgccgtgacccagaatcgaaccgacttccataccgccggtactctgccacgcagactgacctgccaaaatatcgtctttcgtcatcagcactttgcctgatgcggaaacgacctgttcggggtaaggcggggctttcttataaacctcgctgcccatatagccaagaatggtaaagcataccgccagaacggcaaacagcaagtaccacagcttcttgtactgtcccat

SEQ ID NO: 15 - DNA, 2256 bp, Neisseria meningitidis
ttatttgtcgcggccgaatacgattttagtggcttggatggcaacgcagattgcaccgccgataaagaccaagtcagctgccgtacgtacccaacgcaaggtgtcgaggatttccatttgcaggaactcttcgctgcgtgcataccacaggccgtgcgtgatggaggcgtatgcctgaatcacgccaaccggcaacaggctgatggcaatcataccgaccaagccgccgttgagcagccagaagccccaagtcatcagtttgtcgtcaaactgcgcgttcggtttcaaataacgggcaaccagcaatacgaagcccaatgccaagaaaccgtacacaccgaacaaggcggcgtgcgcatgaacagcagaagtattcaaaccttggatatagaacagggaaatcggcgggttaatcaggaaaccgaatacaccggcaccgatcatattccaaaaggcgactgccacgaagcacatcagcggccaacgcaggcgtttcgcccagtcggacaggtgttggtaagaccaatgctcgtatgcttcacggcccagcaacaccagcggcacgacttccaaagcggagaagcaggcaccgattgccatagaggcggaggtagagccggagaagtacaggtggtgcagcgtacccggaacgccgcccaacataaagatggcggcagcggccagagtagaagctgtggcggtactgcggcggacaaagcccatattgtagaagacaaaggcaaaagcggcagtggcaaatacttcaaagaagccttccacccacaggtgaaccacccaccaacgccagtattccattacggcaatcggggatttctcgccatagaacagtcccggtgcgtagaatacgcccacaccgaccatagaagcgacaaagattgccagcaggtttttgtccacgcctttttctttgaaggcggaaaccgtgcaacgcaacatcaggaacagccacaacagcaggccgaccatcagcaggagttgccagaaacgtcccaaatcgaggtattcgtaaccttggtgtccgaaccagaagttaaattccgggggaaggatgtgcgtcaacgcgaagaagttgcccgcgtaagagccgccgaccacgatgaagagggcgatatagaggaagttcacgcctgcacgttggaacttgggatctttgccgccgttgacaatcggcgcaaggaacaaacctgccgtcaaaaagccggttgcaatccagaagatggcggattggatgtgccaagtacgggtcagggcgtaagggaaccagtcggacatttcaaagcccaacgcctcgtcaatgccgtagaaaccctgaccttcgacggtgtagtgcgcggtcagaccgcccagcaatacttgtaccacaaacagggcgaccgtcaggaagacgtatttgcccaatgctttttgcgaaggggtcagttggattttggaaatcgggtcttcagacggcacttccacttcctcgtgtttggtcaggaaggaataaccccacatcaacaaaccgatgcccatcagcagaagaacaacgctggtgaatgaccacatatagttttcagtggtcggtacgttgttgatcaaaggctcgtgcggccagttgttggtataagtgaacgtctcgtcaggacggttggtcgaagcagaccaagaagtccagaagaagaagtcgaacagtttttcacgcgcttcttggcttggcaatgtgttgtttttcattgcaaagtgttcgcgggtggtttgcagcgcgggatcatcgccgtaaacaccgtggtaataaggcaggatgctttcgatggctttcacgcgcgtatcgctgatgacgacgctgccgtcttctttaatacggctttggttgcggtattcgtcagccagtcgggttttcagaacggcttgttcttcaggggaaacttcatcgaattttttgccgtaagtctgttgcgcggtcaaatccaactatgcaaccaactcacgatgcagccagtcggccgtccagtccggagcctgatatgcgccgtgacccagaatcgaaccgacttccataccgccggtactctgccacgcagactgacctgccaaaatatcgtctttcgtcatcagcactttgcctgatgcggaaacgacctgttcagggtaaggcggggcttttttgtaaacctcgctgcccatatagccaagaatggtaaagcataccgccagaacggcaaacagcaagtaccatagcttcttgtactgtcccat

SEQ ID NO: 16 - DNA, 2256 bp, Neisseria lactamica
ttatttgtcccggccgaatacgattttagtggcttggatggcaacgcagattgcaccgccgataaagatcaagtcggcggcagtacgaacccagcgcagggtgtcgaggatttccatttgcaggaactcttcgctgcgggcataccacagaccgtgcgtgatggaggcgtatgcctgaatcgcgccgacaggcagcaggctgatggcaatcataccgaccaagccgccgttgagcaaccagaagccccaagtcatcagtttgtcgtcaaactgcgcgttcggtttcaagtagcgtgcaaccagcaatacgaagcccaatgccaagaaaccgtacacaccgaacaaggcggcgtgcgcgtgaacggcagaagtgttcaaaccttggatatagaacagggaaatcggcggattgatcaggaagccgaatacgccggcaccgatcatattccaaaaggcgactgccacgaagcacatcagcggccaacgcaggcgtttcgcccagtcggacagatgttggtaagaccaatgctcgtatgcttcacgacccagcaacaccagcggcacgacttccaaagcggagaagcaggcgccgattgccatagaggcggaggtagagccggagaagtacaggtggtgcagcgtaccgggaacgccgcccaacataaagatggcggcagcagccaaagtggaggcagtggcggtactgcggcggacaaagcccatattgtagaagacaaaggcaaaggcggcagtggcaaatacttcaaagaagccttccacccacaggtgaaccacccaccagcgccagtattccattacggcaatcggggatttttcgccatagaacagtcccggtgcgtagaacacgcccacaccgaccatagaagcgacaaagattgccagcaggtttttatccacgcctttttctttgaaggcggaaaccgtgcagcgcaacatcaggaacagccacaacagcaggccgaccatcaacaggagttgccagaaacgtcccaaatcgaggtattcgtaaccttggtgtccgaaccagaagttaaattcggggggaaggatgtgcgtcaacgcgaagaagttgcccgcgtaagaaccgccgaccacgataaacagggcgatatagaggaagttcacgcctgcacgttggaacttgggatctttgccgccgttgacaatcggcgcgaggaacaaacctgccgtcaaaaagccggttgcaatccagaagatggcggattggatgtgccaagtacgggtcagggcataggggaaccagtcggacatttcaaagcccaacgcctcatcgatgccgtagaaaccctgaccttcgacggtgtagtgcgcggtcagaccgcccagcaatacttgtaccacaaacagggcgaccgtcaggaagacgtatttgcccaatgctttttgcgaaggggtcagttggattttggaaatcggatcttcagccgggatttccacttcctcgtgtttggtcaggaaggaataaccccacatcagcaaaccgatgcccatcagcagaagaacaacgctggtaaacgaccacatatagttttcagtggtcggcacattgttgatcaaaggctcgtgcggccagttgttggtgtaggtaaagacctcgccgggacggttggtcgaagcagaccaagaagtccaaaagaagaagtcgaacagtttttcacgcgcttcttggcttggcaatgtgttgtttttcattgcaaagtgttcgcgggtggtttgcagcttgggatcgtcgccgtaaacgccgtggtaataaggcaggatgctttcgatggctttcacgcgcgtatcgctgatgacgacgctgccgtcttctttcacacggctttggttgcggtattcgtcagccaggcgggttttcagaacggcttgttcttcaggggaaacttcatcaaattttttgccgtaagtctgttgcgcggtcaaatccaaccaggcagacaactcacgatgcagccagtcggccgtccagtccggagcctgatatgcgccgtgacccaaaatcgaaccgacttccataccgccggtactctgccatgcagactgacctgccaaaatatcgtctttcgtcatcagcactttgcctgatgcggaaacgacctgttcgggataaggcggggctttcttataaacctcgctgcccatatagccgagaatggtaaagcataccgccagaacggcgaacagcaagtaccacagcttcttatactgtcccat